(19) United States
(12) Patent Application Publication
(10) Pub. No.: US 2005/0234065 A1
(43) Pub. Date: Oct. 20, 2005

(54) Dipeptidyl peptidase-IV inhibitors

(75) Inventors: Bernard Hulin, Essex, CT (US); Janice C. Parker, Ledyard, CT (US); David W. Piotrowski, Groton, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/102,394

(22) Filed: Apr. 8, 2005

Related U.S. Application Data
(60) Provisional application No. 60/562,583, filed on Apr. 14, 2004.
(60) Provisional application No. 60/659,518, filed on Mar. 7, 2005.

(51) Int. Cl. A61K 31/519; A61K 31/498; C07D 471/02; C07D 491/02; C07D 498/02

(52) U.S. Cl. 514/249; 514/365; 514/423; 514/303; 514/301; 514/265.1; 514/302; 514/373; 544/330; 544/281; 546/114; 546/115; 546/118; 548/537; 548/200

(57) ABSTRACT

The invention provides compounds of Formula (I) 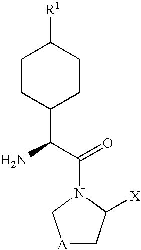
or prodrugs thereof, or pharmaceutically acceptable salts of said compounds or prodrugs, or solvates of said compounds, prodrugs or salts, wherein A, N, X and R¹ are as defined herein; pharmaceutical compositions thereof; and methods of using the pharmaceutical compositions for the treatment of diseases, including Type 2 diabetes, Type 1 diabetes, impaired glucose tolerance, hyperglycemia, metabolic syndrome (syndrome X and/or insulin resistance syndrome), glucosuria, metabolic acidosis, arthritis, cataracts, diabetic neuropathy, diabetic nephropathy, diabetic retinopathy, diabetic cardiomyopathy, obesity, conditions exacerbated by obesity, hypertension, hyperlipidemia, atherosclerosis, osteoporosis, osteopenia, frailty, bone loss, bone fracture, acute coronary syndrome, short stature due to growth hormone deficiency, infertility due to polycystic ovary syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome; short bowel syndrome; and the prevention of disease progression in Type 2 diabetes.

### FIELD OF THE INVENTION

The present invention relates to compounds which inhibit the enzyme dipeptidyl peptidase-IV (hereinafter “DPP-IV”), pharmaceutical compositions comprising said compounds and the use of said compounds and pharmaceutical compositions to treat diabetes and to treat diseases that are associated with proteins that are subject to processing by DPP-IV.

### BACKGROUND OF THE INVENTION

DPP-IV (EC 3.4.14.5) is a serine protease that preferentially hydrolyzes an N-terminal dipeptide from proteins having proline or alanine in the 2 position. DPP-IV is believed to be involved in diabetes, glucose tolerance, obesity, appetite regulation, lipidemia, osteoporosis, neuropeptide metabolism and T-cell activation, among others.

DPP-IV has been implicated in the control of glucose homeostasis because its substrates include the incretin peptides glucagon-like peptide 1 (GLP-1) and gastric inhibitory polypeptide (GIP). Cleavage of the N-terminal amino acids from these peptides renders them functionally inactive. GLP-1 has been shown to be an effective anti-diabetic therapy in Type 2 diabetic patients and to reduce the meal-related insulin requirement in Type 1 diabetic patients. GLP-1 and/or GIP are believed to regulate satiety, lipidemia and osteogenesis. Exogenous GLP-1 has been proposed as a treatment for patients suffering from acute coronary syndrome, angina and ischemic heart disease.

Administration of DPP-IV inhibitors in vivo prevents N-terminal degradation of GLP-1 and GIP, resulting in higher circulating concentrations of these peptides, increased insulin secretion and improved glucose tolerance. On the basis of these observations, DPP-IV inhibitors are regarded as agents for the treatment of Type 2 diabetes, a disease in which glucose tolerance is impaired. In addition, treatment with DPP-IV inhibitors prevents degradation of Neuropeptide Y (NPY), a peptide associated with a variety of central nervous system disorders, and Peptide YY which has been linked to gastrointestinal conditions such as ulcers, irritable bowel disease and inflammatory bowel disease.

In spite of the early discovery of insulin and its subsequent widespread use in the treatment of diabetes, and the later discovery of and use of sulfonylureas (e.g. chlorpropamide, tolbutamide, acetohexamide), biguanides (e.g., phenformin, metformin), and thiazolidinediones (e.g., rosiglitazone, pioglitazone) as oral hypoglycemic agents, the treatment of diabetes remains less than satisfactory.

The use of insulin, necessary in Type 1 diabetic patients and about 10% of Type 2 diabetic patients in whom currently available oral hypoglycemic agents are ineffective, requires multiple daily doses, usually by self-injection. Determination of the appropriate dosage of insulin necessitates frequent estimations of the glucose concentration in urine or blood. The administration of an excess dose of insulin causes hypoglycemia, with consequences ranging from mild abnormalities in blood glucose to coma, or even death.

Treatment of Type 2 diabetes usually comprises a combination of diet, exercise, oral agents, and in more severe cases, insulin. However, the clinically available hypoglycemics can have side effects that limit their use. A continuing need for hypoglycemic agents, which may have fewer side effects or succeed where others fail, is clearly evident.

Poorly controlled hyperglycemia is a direct cause of the multiplicity of complications (cataracts, neuropathy, nephropathy, retinopathy, cardiomyopathy) that characterize advanced Type 2 diabetes. In addition, Type 2 diabetes is a co-morbid disease that frequently confounds hyperlipidemia, atherosclerosis and hypertension, adding significantly to the overall morbidity and mortality attributable to those diseases.

Epidemiological evidence has firmly established hyperlipidemia as a primary risk factor for cardiovascular disease (“CVD”) due to atherosclerosis. CVD is especially prevalent among diabetic subjects, at least in part because of the existence of multiple independent risk factors such as glucose intolerance, left ventricular hypertrophy and hypertension in this population. Successful treatment of hyperlipidemia in the general population, and in diabetic subjects in particular, is therefore of exceptional medical importance.

Hypertension (or high blood pressure) is a condition that can occur in many patients in whom the causative agent or disorder is unknown. Such “essential” hypertension is often associated with disorders such as obesity, diabetes and hypertriglyceridemia, and it is known that hypertension is positively associated with heart failure, renal failure and stroke. Hypertension can also contribute to the development of atherosclerosis and coronary disease. Hypertension, together with insulin resistance and hyperlipidemia, comprise the constellation of symptoms that characterize metabolic syndrome, also known as insulin resistance syndrome (“IRS”) and syndrome X.

Obesity is a well-known and common risk factor for the development of atherosclerosis, hypertension and diabetes. The incidence of obesity and hence of these diseases is increasing worldwide. Currently few pharmacological agents are available that reduce adiposity effectively and acceptably.

Osteoporosis is a progressive systemic disease characterized by low bone density and microarchitectural deterioration of bone tissue, with a consequent increase in bone fragility and susceptibility to fracture. Osteoporosis and the consequences of compromised bone strength are a significant cause of frailty, and of increased morbidity and mortality.

Heart disease is a major health problem throughout the world. Myocardial infarctions are a significant source of mortality among those individuals with heart disease. Acute coronary syndrome denotes patients who have or are at high risk of developing an acute myocardial infarction (MI). Though there are therapies available for the treatment of diabetes, hyperglycemia, hyperlipidemia, hypertension, obesity and osteoporosis there is a continuing need for alternative and improved therapies. Various indications for dipeptidyl peptidase inhibitors are discussed in the following review articles: Augustyns et al., Curr. Medicinal Chem. 6, 311 (1999); Ohnuki et al., Drugs of the Future 24, 665-670 (1999); Villhauer et al., Annual Reports in Medicinal Chemistry 36, 191-200 (2001); Drucker, Expert Opin. Invest. Drugs, 2003, 12, 87-100; Wiedeman & Trevillyan, Curr. Opin. Invest. Drugs 2003, 4, 412-420.

Compounds that inhibit DPP-IV have been recently developed, such as those disclosed in International Application WO02/076450. However, many of these compounds are predicted to have poor gastrointestinal permeability, such as through use of Madin-Darby Canine Kidney Cells (MDCK) Permeability Assays, which may result in a low compound bioavailability when administered orally.

Therefore, what is needed is an orally administered DPP-IV inhibitor compound that has equivalent or better DPP-IV inhibitory activity and improved gastrointestinal permeability.

### SUMMARY OF INVENTION

The present invention relates to compounds having the structure of Formula (I) 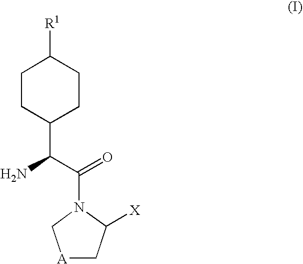
or a prodrug thereof, or a pharmaceutically acceptable salt of said compound or prodrug, or a solvate of said compound, prodrug or salt, wherein:
X is H or —CN;
A is CH₂, CHF, CF₂ or S(O)ₙ;
n is 0, 1 or 2;
R¹ is —NR²R³, Het^{(I)}, or Het^{(II)};
R² is —C(O)R⁴, —SO₂R⁴, —C(O)NHR⁴, or —COOR⁴;
R³ is H, C₁₋₆alkyl, or C₃₋₈cycloalkyl;
R⁴ is selected from the group consisting of
   (a) Het^{(I)}-C₀₋₆alkylenyl-,
   (b) Het^{(II)}-C₀₋₆alkylenyl-,
   (c) R⁵OC(O)N(R⁶)-C₁₋₆alkylenyl-,
   (d) R⁵C(O)N(R⁶)-C₁₋₆alkylenyl-,
   (e) phenyl-C₀₋₆alkylenyl-amino-C₀₋₆alkylenyl-,
   (f) phenylsulfonyl-C₁₋₆alkylenyl-,
   (g) phenylthio-C₁₋₆alkylenyl-,
   (h) naphthyloxy-C₁₋₆alkylenyl-, and
   (i) C₃₋₈cycloalkyl- wherein said C₃₋₈cycloalkyl is optionally substituted with C₁₋₆alkyl, C₁₋₆alkoxy, hydroxy, halo, or phenyl optionally substituted with one to three halo; OKHet^{(I)} is oxazolidinyl, 2,3-dihydro-1H-pyrrolo[3,4-b]pyridyl, 6,7-dihydro-5H-pyrrolo[3,4-b]pyrazinyl, 6,7-dihydro-5H-pyrrolo[3,4-b]pyridyl, 2,3-dihydro-1H-pyrrolo[3,4-c]pyridyl, 5,6-dihydro-4H-thieno[2,3-c]pyrrolyl, pyrrolo[1,2-c]pyrimidyl, 1H-pyrrolo[2,3-c]pyridyl, 2,3-dihydro-furo[2,3-c]pyridyl, pyrrolo [1,2-a]pyrazinyl, thieno[3,2-c]pyridyl, furo[2,3-c]pyridyl, thieno[2,3-c]pyridyl, furo[3,2-c]pyridyl, 1,1-dioxo-1,3-dihydro-1λ⁶-benzo[d]isothiazol-2-yl, or triazinyl, wherein Het^{(I)} is optionally and independently substituted with from one to three substituents selected from the group consisting of halo, hydroxy, oxo, C₁₋₆alkyl, C₁₋₆alkenyl, C₁₋₆alkynyl, C₁₋₆alkoxy, phenylC₀₋₆alkylenyl-, benzyloxycarbonyl-, and C₁₋₆ alkoxycarbonyl-;
R⁵ is C₁₋₆alkyl or phenylC₀₋₆alkylenyl-;
R⁶ is H, C₁₋₆alkylenyl, or C₃₋₈cycloalkyl;
Het^{(II)} is furanyl, dihydrofuranyl, tetrahydrofuranyl, pyranyl, dihydropyranyl, tetrahydropyranyl, thienyl, dihydrothienyl, tetrahydrothienyl, pyridyl, pyrimidyl, pyrazinyl, pyrrolidinyl, piperidinyl, imidazolyl, pyrazolyl, pyrrolyl, oxazolyl, isoxazolyl, thiazolyl, thiazolidinyl, thiadiazolyl, triazolyl, azetidinyl, dioxanyl, morpholinyl, thiomorpholinyl, imidazolidinyl, thiazolidinyl or a benzo-fused analogue of said Het, wherein Het^{(II)} is substituted with one to three substitutents independently selected from the group consisting of hydroxy, aminocarbonyl-, C₁₋₆alkylaminocarbonyl-, phenyl-C₁₋₆alkylamino carbonyl-, cyano, phenyl-C₁₋₆alkylenylamino-, benzylidene, benzyloxy-C₁₋₆alkylenyl-, benzyloxycarbonyl-, C₁₋₆alkoxycarbonyl-, nitro, and —NR⁷R⁸, and wherein Het^{(II)} is optionally substituted with one to three substituents independently selected from the group consisting of halo, trifluoromethyl, oxo, C₁₋₆alkyl, C₁₋₆alkoxy, C₁₋₆alkylphenyl-, or C₁₋₆alkylcarbonyl; and
R⁷ and R⁸ are each independently selected from H or C₁₋₆alkyl, or R⁷ and R⁸ may be taken together with the N atom to which they are attached to form a three to seven membered saturated, partially unsaturated, or unsaturated heterocyclic ring, wherein said heterocyclic ring optionally comprises an additional one to three heteroatoms selected from O, S, and N.

The present invention also relates to a pharmaceutical composition comprising a therapeutically effective amount of a compound of the present invention, or a prodrug thereof, or a pharmaceutically acceptable salt of the compound or prodrug, or a solvate of the compound, prodrug or salt, and a pharmaceutically acceptable carrier, vehicle, diluent or excipient.

The present invention further relates to a method of treating diabetes comprising administering to a mammal in need of such treatment a therapeutically effective amount of a compound of the present invention, or a prodrug thereof, or a pharmaceutically acceptable salt of the compound or of the prodrug, or a solvate of the compound, prodrug or salt. Preferably, the type of diabetes treated is Type 2 diabetes.

The present invention additionally relates to a method of treating a condition mediated by dipeptidyl peptidase-IV in a mammal comprising administering to said mammal in need of such treatment a therapeutically effective amount of a compound of the present invention, or a prodrug thereof, or a pharmaceutically acceptable salt of said compound or prodrug, or a solvate of said compound, prodrug or salt.

The compounds, and pharmaceutical compositions, of the present invention are useful for the treatment of diabetes, preferably Type 2 diabetes.

The compounds, and pharmaceutical compositions, of the present invention are also useful for the treatment of dipeptidyl peptidase-IV related conditions which include, but are not limited to, Type 2 diabetes; Type 1 diabetes, impaired glucose tolerance, hyperglycemia, metabolic syndrome (syndrome X and/or insulin resistance syndrome), glucosuria, metabolic acidosis, arthritis, cataracts, diabetic neuropathy, diabetic nephropathy, diabetic retinopathy, diabetic cardiomyopathy, obesity, conditions exacerbated by obesity, hypertension, hyperlipidemia, atherosclerosis, osteoporosis, osteopenia, frailty, bone loss, bone fracture, acute coronary syndrome, short stature due to growth hormone deficiency, infertility due to polycystic ovary syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome; short bowel syndrome; and the prevention of disease progression in Type 2 diabetes.

### DETAILED DESCRIPTION

The terms used to describe the present invention have the following meanings herein.

The phrase “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The carbon atom content of the various hydrocarbon-containing moieties herein may be indicated by a prefix designating the minimum and maximum number of carbon atoms in the moiety, for example, the prefixes (Cₐ-C_{b})alkyl, and C_{a-b}alkyl, indicate an alkyl moiety of the integer “a” to “b” carbon atoms, inclusive. Thus, for example, (C₁-C₆)alkyl and C₁₋₆alkyl refer to an alkyl group of one to six carbon atoms inclusive.

The term “alkyl” as used herein, means a saturated monovalent straight or branched aliphatic hydrocarbon radical, wherein the number of carbon atoms may be defined in a parenthetical where the term is used. Examples of alkyl groups include methyl, ethyl, propyl, butyl, and the like.

The term “alkoxy” refers to straight or branched, monovalent, saturated aliphatic chains of carbon atoms bonded to an oxygen atom that is attached to a core structure. Examples of alkoxy groups include methoxy, ethoxy, propoxy, butoxy, iso-butoxy, tert-butoxy, and the like.

The term “cycloalkyl” denotes a saturated monocyclic or bicyclic cycloalkyl group. Cycloalkyl groups may be optionally fused to aromatic hydrocarbons such as benzene to form fused cycloalkyl groups, such as indanyl and the like. Examples of cycloalkyl groups include cyclopropyl, cyclobutyl, cyclopentyl, cyclohexyl, cycloheptyl, and the like.

The term “halogen” or “halo” represents chloro, bromo, fluoro, and iodo atoms and substituents.

The term “heterocyclyl” or “heterocycle” denotes a saturated monocyclic or polycyclic cycloalkyl group, in which at least one of the carbon atoms is replaced with a heteroatom such as nitrogen, oxygen, or sulfur. If the heterocyclyl contains more than one heteroatom, the heteroatoms may be the same or different. A cyclic group may be bonded to another group in more than one way. If no particular bonding arrangement is specified, then all possible arrangements are intended. For example, the term “pyridyl” includes 2-, 3-, or 4-pyridyl.

The term “oxo”, means a carbonyl group formed by the combination of a carbon atom and an oxygen atom.

The term “substituted” means that a hydrogen atom on a molecule has been replaced with a different atom or molecule. The atom or molecule replacing the hydrogen atom is denoted as a “substituent.” The symbol “—” represents a covalent bond.

The phrase “inert solvent” refers to a solvent, or mixture of solvents, that does not interact with starting materials, reagents, intermediates, or products in a manner that adversely affects their desired properties.

The terms “treating”, “treated”, or “treatment” as employed herein includes preventative (e.g., prophylactic), palliative, and curative uses or results.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” is an individual animal that is a member of the taxonomic class Mammalia. The class Mammalia includes, for example, humans, monkeys, chimpanzees, gorillas, cattle, swine, horses, sheep, dogs, cats, mice and rats. In the present invention, the preferred mammal is a human.

The compounds of the present invention contain at least three stereogenic centers. Consequently, those skilled in the art will appreciate that all stereoisomers (e.g., enantiomers and diasteroisomers, and racemic mixtures thereof) of the compounds illustrated and discussed herein are within the scope of the present invention. For example, the compounds of Examples 1-7 all contain cyclohexane in the cis stereoconfiguration.

Preferably, the compounds of Formula (I) of the present invention all contain the 1,4-substituents of the cyclohexane ring in the trans stereoconfiguration, such as is shown below in two different representations in Figure (IA) 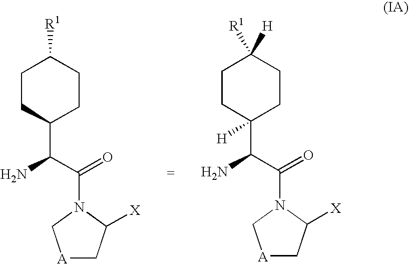
or as is further exemplified by the compounds of Examples 8-62 herein.

More preferably, the compounds of the present invention have the structure of Formula (IA) wherein:
X is H or —CN;
A is CH₂, CHF, CF₂ or S;
R¹ is —NR²R³, Het^{(I)}, or Het^{(II)};
R² is —C(O)R⁴;
R³ is H;
R⁴ is selected from the group consisting of
   (a) Het^{(I)}-C₀₋₆alkylenyl-,
   (b) Het^{(II)}-C₀₋₆alkylenyl-, and
   (c) R⁵OC(O)N(R⁶)-C₁₋₆alkylenyl-; OK
Het^{(I)} is oxazolidinyl, 6,7-dihydro-5H-pyrrolo[3,4-b]pyrazinyl, 6,7-dihydro-5H-pyrrolo[3,4-b]pyridyl, 2,3-dihydro-1H-pyrrolo[3,4-c]pyridyl, 5,6-dihydro-4H-thieno[2,3-c]pyrrolyl, pyrrolo[1,2-c]pyrimidyl, 1H-pyrrolo[2,3-c]pyridyl, 2,3-dihydro-furo[2,3-c]pyridyl, pyrrolo[1,2-a]pyrazinyl, thieno[3,2-c]pyridyl, furo[2,3-c]pyridyl, thieno[2,3-c]pyridyl, furo[3,2-c]pyridyl, or 1,1-dioxo-1,3-dihydro-1λ⁶-benzo[d]isothiazol-2-yl, wherein Het^{(I)} is optionally and independently substituted with from one to three substituents selected from the group consisting of halo, hydroxy, oxo, C₁₋₆alkyl, C₁₋₆alkenyl, C₁₋₆alkynyl, C₁₋₆alkoxy, phenylC₀O₆alkylenyl-, benzyloxy-carbonyl-, and C₁₋₆alkoxycarbonyl-;
R⁵ is phenylC₀₋₆alkylenyl-;
R⁶ is H or C₁₋₆alkylenyl;
Het^{(II)} is pyridyl, pyrazinyl, pyrrolidinyl, pyrazolyl, imidazolidinyl or isoindole, wherein Het^{(II)} is substituted with one to three substitutents independently selected from the group consisting of hydroxy, aminocarbonyl-, C₁₋₆alkylaminocarbonyl-, phenyl-C₁₋₆alkylaminocarbonyl-, cyano, phenyl-C₁₋₆alkylenylamino-, benzylidene, benzyloxy-C₁₋₆alkylenyl-, benzyloxycarbonyl-, C₁₋₆alkoxycarbonyl-, nitro, and —NR⁷R⁸, and wherein Het^{(II)} is optionally substituted with one to three substituents independently selected from the group consisting of halo, trifluoromethyl, oxo, C₁₋₆alkyl, C₁₋₆alkoxy, C₁₋₆alkylphenyl-, or C₁₋₆alkylcarbonyl-; and
R⁷ and R⁸ are each independently selected from H or C₁₋₆alkyl.

For the compounds of Formula (IA), it is preferred that R¹ is —NR²R³.

In the compounds of the present invention, it is more preferred that the compounds of Formula (IA) have the structure of Formula (IB), shown below. 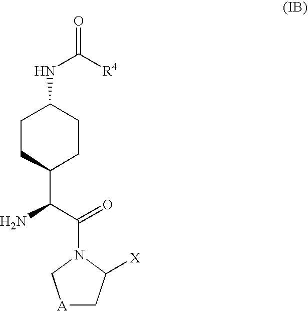
wherein:
X is H or —CN;
A is CH₂, CHF, CF₂ or S;
R⁴ is Het^{(II)}-C₀₋₆alkylenyl-, and
Het^{(II)} is pyridyl, pyrazinyl, pyrrolidinyl, pyrazolyl, imidazolidinyl or isoindole, wherein Het^{(II)} is substituted with one to three substitutents independently selected from the group consisting of hydroxy, aminocarbonyl-, C₁₋₆alkylaminocarbonyl-, phenyl-C₁₋₆alkylaminocarbonyl-, cyano, phenyl-C₁₋₆alkylenylamino-, benzylidene, benzyloxy-C₁₋₆alkylenyl-, benzyloxycarbonyl-,
C₁₋₆alkoxycarbonyl-, nitro, and —NR⁷R⁸, and wherein Het^{(II)} is optionally substituted with one to three substituents independently selected from the group consisting of halo, trifluoromethyl, oxo, C₁₋₆alkyl, C₁₋₆alkoxy, C₁₋₆alkylphenyl-, or C₁₋₆alkylcarbonyl-; and
R⁷ and R⁸ are each independently selected from H or C₁₋₆alkyl.

Preferably, for the compounds of Formula (IB), Het^{(II)} is selected from pyrazinyl and pyridyl, and more preferably, said pyrazinal or pyridyl is substituted with —NR⁷R⁸.

Yet more preferably, in the compounds of Formula (IB), R² is 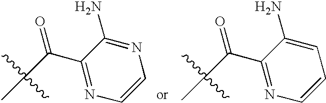
and even more preferably A is S.

In the present invention, the compound (S)-3-amino-pyrazine-2-carboxylic acid [trans-4-(1-amino-2-oxo-2-thiazolidin-3-yl-ethyl)-cyclohexyl]-amide, or a pharmaceutically acceptable salt thereof, is most preferred.

The stereoisomers, of compounds of the present invention, may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired stereoisomer is converted into another chemical entity by one of the separation procedures described above, a further step is required to liberate the desired enantiomeric form. Alternatively, specific stereoisomers may be synthesized by asymmetric synthesis using optically active reagents, substrates, catalysts or solvents, or by converting one stereoisomer into the other by asymmetric transformation.

Certain compounds of Formula (I) may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Formula (I) and mixtures thereof.

Practitioners will appreciate that certain compounds of Formula (I) may exist in tautomeric form, i.e., that an equilibrium exists between two isomers which are in rapid equilibrium with each other. A common example of tautomerism is keto-enol tautomerism, i.e.,

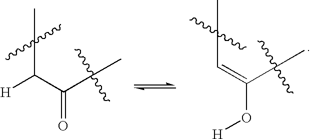

Examples of such compounds of the present invention include, inter alia, hydroxypyridines (pyridones) and hydroxypyrmidines (pyrimidones). In particular, a person skilled in the art will recognize that a hydroxypyridine of the instant invention can exist as two separate tautomers, e.g.,

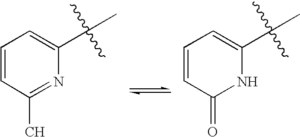

The degree to which one tautomer is present over the other depends upon various factors, including substitution pattern and solvent type. Other examples in accordance with the present invention will be recognized by those skilled in the art. All tautomeric forms of Formula (I) are included within the scope of the claimed invention.

The compounds of the present invention may exist in unsolvated as well as solvated forms with pharmaceutically acceptable solvents such as water, ethanol, and the like, and it is intended that the invention embrace unsolvated forms, solvated forms and mixtures of solvated forms.

Certain compounds of Formula (I) and their salts and solvates may exist in more than one crystal form. Polymorphs of compounds represented by Formula (I) form part of this invention and may be prepared by crystallization of a compound of Formula (I) under different conditions. For example, using different solvents or different solvent mixtures for recrystallization; crystallization at different temperatures; various modes of cooling, ranging from very fast to very slow cooling during crystallization. Polymorphs may also be obtained by heating or melting a compound of Formula (I) followed by gradual or fast cooling. The presence of polymorphs may be determined by solid probe nmr spectroscopy, ir spectroscopy, differential scanning calorimetry, powder X-ray diffraction or such other techniques.

This invention also includes isotopically-labeled compounds, which are identical to those described by Formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, sulfur and fluorine, such as ²H, ³H, ¹³C, ¹⁴C, ¹⁵N, ¹⁸O, ¹⁷O, ³⁵S, ³⁶Cl, ¹²⁵I, ¹²⁹I, and ¹⁸F respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of Formula (I) of this invention and prodrugs thereof can generally be prepared by carrying out the procedures disclosed in the schemes and/or in the Examples below, by substituting a readily available isotopically labeled reagent for a non-isotopically labeled reagent.

Pharmaceutically acceptable salts, as used herein in relation to compounds of the present invention, include pharmaceutically acceptable inorganic and organic salts of said compound. These salts can be prepared in situ during the final isolation and purification of a compound, or by separately reacting the compound or prodrug with a suitable organic or inorganic acid and isolating the salt thus formed. Representative salts include, but are not limited to, the hydrobromide, hydrochloride, hydroiodide, sulfate, bisulfate, nitrate, acetate, trifluoroacetate, oxalate, besylate, palmitate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate and laurylsulphonate salts, and the like. See, e.g., Berge, et al., J. Pharm. Sci., 66, 1-19 (1977).

The compounds of the present invention may be isolated and used per se or in the form of their pharmaceutically acceptable salts or solvates. In accordance with the present invention, compounds with multiple basic nitrogen atoms can form salts with varying number of equivalents of acid. It will be understood by practitioners that all such salts are within the scope of the present invention.

A prodrug of a compound of Formula (I) may be one formed in a conventional manner with a functional group of the compound, such as with an amino, hydroxy or carboxy group. The term “prodrug” means a compound that is transformed in vivo to yield a compound of Formula (I) or a pharmaceutically acceptable salt or solvate of the compound. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of the present invention incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY′ wherein Y′ is H, (C₁-C₆)alkyl or benzyl, —C(OY₀)Y₁ wherein Y₀ is (C₁-C₄)alkyl and Y₁ is (C₁-C₆)alkyl, carboxy(C₁-C₆)alkyl, amino(C₁-C₄)alkyl or mono-N- or di-N,N-(C₁-C₆)alkylaminoalkyl, —C(Y₂)Y₃ wherein Y₂ is H or methyl and Y₃ is mono-N- or di-N,N-(C₁-C₆)alkylamino, morpholino, piperidin-1-yl or pyrrolidin-1-yl.

Similarly, if a compound of the present invention contains an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

In general, the compounds of Formula (I) of this invention may be prepared by methods that include processes known in the chemical arts, particularly in light of the description contained herein. Certain processes for the manufacture of the compounds of Formula (I) of this invention are illustrated by the following reaction schemes. Other processes are described in the experimental section. Some of the starting compounds for the reactions described in the schemes and Examples are prepared as illustrated herein. All other starting compounds may be obtained from general commercial sources, such as Sigma-Aldrich Corporation, St. Louis, Mo.

Some of the compounds of Formula (I), wherein R² and R³ are defined above, may be prepared by the synthetic sequence illustrated in Scheme 1, shown below.

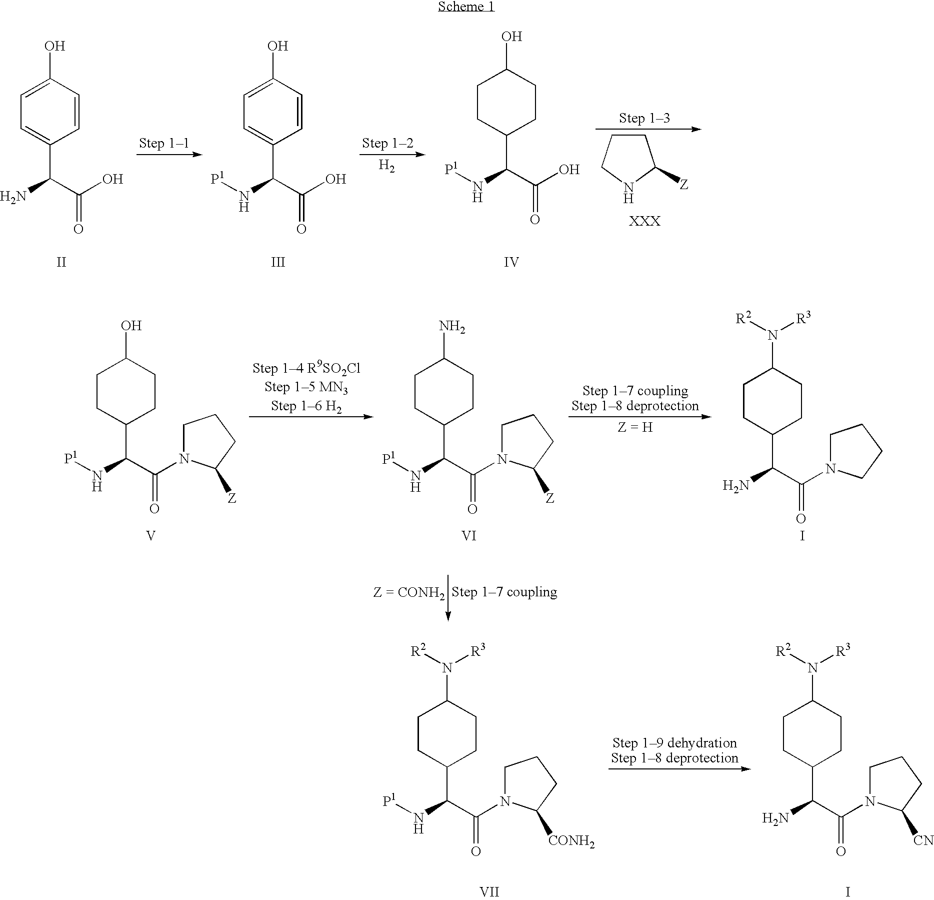

Step 1-1 comprises protecting p-hydroxyphenylglycine II with a group P¹ that is inert to the conditions of steps 1-2 to 1-7 and step 1-9. A compound of Formula (I)II is produced. P¹ is preferably a nitrogen-protecting group, and may include, for example, tert-butoxycarbonyl (“Boc”), benzloxycarbonyl (“Cbz”), and fluorenylmethoxycarbonyl (“Fmoc”). This reaction is readily accomplished by dissolving a compound of Formula (I)I in an inert solvent such as dioxane or THF. To the resulting solution is added an appropriate reagent, e.g. di(tert-butyl)carbonate or benzyl chloroformate. The reaction is conducted at a suitable temperature, such as 0 to 80° C., preferably at room temperature, for a suitable time, such as 1 to 24 hours, for example 16 hours, in the optional presence of a base (e.g. triethylamine or pyridine). Other examples of nitrogen-protecting groups are described in “Protective Groups in Organic Synthesis”, 2^{nd} Ed., P. G. M. Wuts and T. W. Greene, including page 315, incorporated herein by reference.

Step 1-2 consists of a hydrogenation at a suitable pressure, such as at 30-60 psi, in the presence of a catalyst such as platinum oxide, Raney Nickel or rhodium at a suitable temperature, such as between 20 and 100° C., for a suitable time, such as 3 to 48 hours. The product IV is isolated by filtering the catalyst through diatomaceous earth and evaporating the solvent. Suitable solvents include ethanol, and ethyl acetate. It will be recognized by those skilled in the art that steps 1-1 and 1-2 can be inverted such that the hydrogenation step is performed before the nitrogen protection step.

Step 1-3 consists of coupling a compound of Formula (I)V with a pyrrolidine. This coupling reaction is readily accomplished by dissolving a compound of Formula (I)V and an optionally substituted pyrrolidine XXX (wherein the substituent Z includes any suitable group, for example hydrogen or CONH₂) in a reaction inert solvent. To the resulting solution is added a coupling agent (e.g. 1-(-3-dimethylaminopropyl)-3-ethylcarbodiimide hydrochloride) in the optional presence of a base (e.g. triethylamine or pyridine) and an optional adjuvant (e.g. hydroxybenzotriazole, azahydroxybenzotriazole). Other suitable coupling agents may be utilized, such as O-(7-azabenzotriazol-1-yl)-N,N,N′,N′-tetramethyluronium hexafluorophosphate (hereinafter “HATU”), dicyclohexylcarbodiimide, 2-ethoxy-1-ethoxycarbonyl-1,2-dihydroquinoline, carbonyldiimidazole or diethylphosphorylcyanide. The coupling is conducted in an inert solvent, preferably an aprotic solvent. The reaction is conducted at a suitable temperature, such as 0 to 50° C., for a suitable time, such as 1 to 24 hours, for example 16 hours. Suitable solvents include, for example, acetonitrile, dichloromethane, dimethylformamide, and chloroform. For a discussion of other conditions useful for coupling carboxylic acids see Houben-Weyl, Vol XV, part II, E. Wunsch, Ed., G. Theime Verlag, (1974), Stuttgart; those described in M. Bodansky, Principles of Peptide Synthesis, Springer-Verlag Berlin (1984); and, those described in The Peptides: Analysis, Synthesis and Biology (ed. E. Gross and J. Meienhofer), vols 1-5 (Academic Press NY 1979-1983). The text of the above references is incorporated herein by reference. It will be recognized by those skilled in the art that other amines can be used in the place of pyrrolidines to prepare other compounds of Formula (I) described in this invention, such that, preferably, any functionality on the amine is inert to the reaction conditions in steps 1-4 to 1-8.

The reaction is generally conducted at ambient pressure and temperature until the starting materials are no longer present as determined by thin layer chromatography or other analytical techniques well known to those skilled in the art. The coupled product of Formula V may be isolated according to methods well known to those skilled in the art.

Steps 1-4 to 1-6 consist in the replacement of the hydroxyl group of V with an amino group with inversion of stereochemistry. A typical sequence for this transformation includes the activation of the hydroxyl group to an alkyl sulfonate, followed by reaction with a metal azide and hydrogenation. Step 1-4 proceeds by reacting formula V with a sulfonyl chloride R⁹SO₂Cl in an inert solvent (e.g. dichloromethane) in the presence of a base, wherein R⁹ may be selected from any suitable group, such as, for example, methyl, phenyl, or toluyl. Combinations of methanesulfonyl chloride t triethylamine and p-toluenesulfonyl chloride/pyridine are particularly effective. If the base chosen is pyridine it can be used as the solvent. The reaction is conducted at a suitable temperature, such as 0 to 80° C., preferably at room temperature, for a suitable time, such as 1 to 24 hours, for example 16 hours.

In step 1-5 the product of step 1-4 and the metal azide MN₃, wherein M is a monovalent metal such as lithium or sodium, are heated together at a suitable temperature, such as 50-100° C., preferably 65° C., in an inert solvent (e.g. DMF, acetonitrile), and the product is isolated by suitable methods known to those skilled in the art. The reaction is conducted for a suitable time, such as 1 to 24 hours, for example 16 hours.

In step 1-6, the product of step 1-5 is hydrogenated at a suitable pressure, such as 30-60 psi, in the presence of a metal catalyst, such as palladium, platinum oxide, Raney Nickel or rhodium, at a suitable temperature, such as 20-100° C., preferably room temperature, for a suitable time, such as 3 to 24 hours, for example 16 hours. The product VI is isolated by filtering the catalyst through diatomaceous earth and evaporating the solvent. Other suitable methods known to those skilled in the art can be used, such as where transforming the azide function to an amine, triphenylphosphine may be used. It will be clear to those skilled in the art that the method chosen for this transformation must be compatible with the protecting group P¹.

In step 1-7, the amine of formula VI is reacted under suitable conditions, such as those described below in Schemes 2 and 3.

Deprotection step 1-8 is described further herein below.

In step 1-9 the amide of formula VII is converted to a cyano group by dissolving VII in an inert solvent (e.g. dichloromethane) and adding a dehydrating agent in the optional presence of a base. Typical dehydrating agents include, but are not limited to, trifluoroacetic anhydride, phosphorous oxychloride, oxalyl chloride and cyanuric chloride. Optional bases include, but are not limited to, pyridine, triethylamine and diisopropylethylamine. The reaction is carried out at a suitable temperature, such as 0-50° C., preferably 0° C., for a suitable time, such 1 to 24 hours, for example 16 hours.

In step 1-8, deprotection of the products of steps 1-7 or 1-9 is performed. If P¹ is Boc, deprotection may proceed by dissolving the product of step 1-7 or 1-9 in an inert solvent (e.g. ethyl acetate, ether, and dioxane) and cooling to a suitable temperature, such as about 0° C., followed by treatment with gaseous acid (e.g. hydrogen chloride) for a suitable time, such as about 1 minute. The reaction mixture is stirred for a suitable time, such as about 5 minutes to about an hour, and then allowed to reach a suitable temperature, such as room temperature, followed by stirring for an additional suitable amount of time, such as about an additional 30 minutes to about 16 hours. In one embodiment, the reaction mixture is stirred about 15 minutes, allowed to reach room temperature, then stirred an additional 30 minutes. Other suitable conditions include dissolving the product of step 1-7 or 1-9 in trifluoroacetic acid and after a suitable reaction time (e.g. 30 min to 24 hours) removing the excess trifluoroacetic acid under vacuum and triturating the compound in a suitable solvent such as ether. If P¹ is benzyloxycarbonyl, deprotection of the product of step 1-7 or 1-9 may be performed by hydrogenolysis in the presence of suitable catalyst, such as 10% palladium or palladium hydroxide, in a suitable solvent such as ethanol or ethyl acetate at a suitable pressure, such as about 30 psi to about 60 psi, and preferably about 45 psi, for a period of time sufficient to bring the reaction to completion, usually overnight, at a suitable temperature, such as 20-80° C., preferably room temperature. A compound of Formula (I) may then be isolated by filtration of the catalyst over diatomaceous earth and removal of the solvent.

In Scheme 2, shown below, within step 2-1, the amine of formula VI is reacted with a carboxylic acid in the presence of a suitable coupling agent, such as described above for step 1-3, to yield an amide product of formula VIII, wherein R³ is as defined above. One skilled in the art will appreciate that if R³COOH is an N-protected amino acid (e.g. N-carbobenzyloxy-L-hydroxyproline), this coupling may be followed by of the removal of the amino acid protecting group (e.g. hydrogenolysis in the presence of a palladium catalyst if the protecting group is carbobenzyloxy).

In step 2-2 the amine of formula VI is reacted with an isocyanate, R³NCO, wherein R³ is as defined above, in an inert solvent (e.g. dichloromethane, THF) to form a urea IX. The reaction is performed at a suitable temperature, such as 0-50° C., preferably room temperature for a suitable time, such 1 to 24 hours, for example 16 hours.

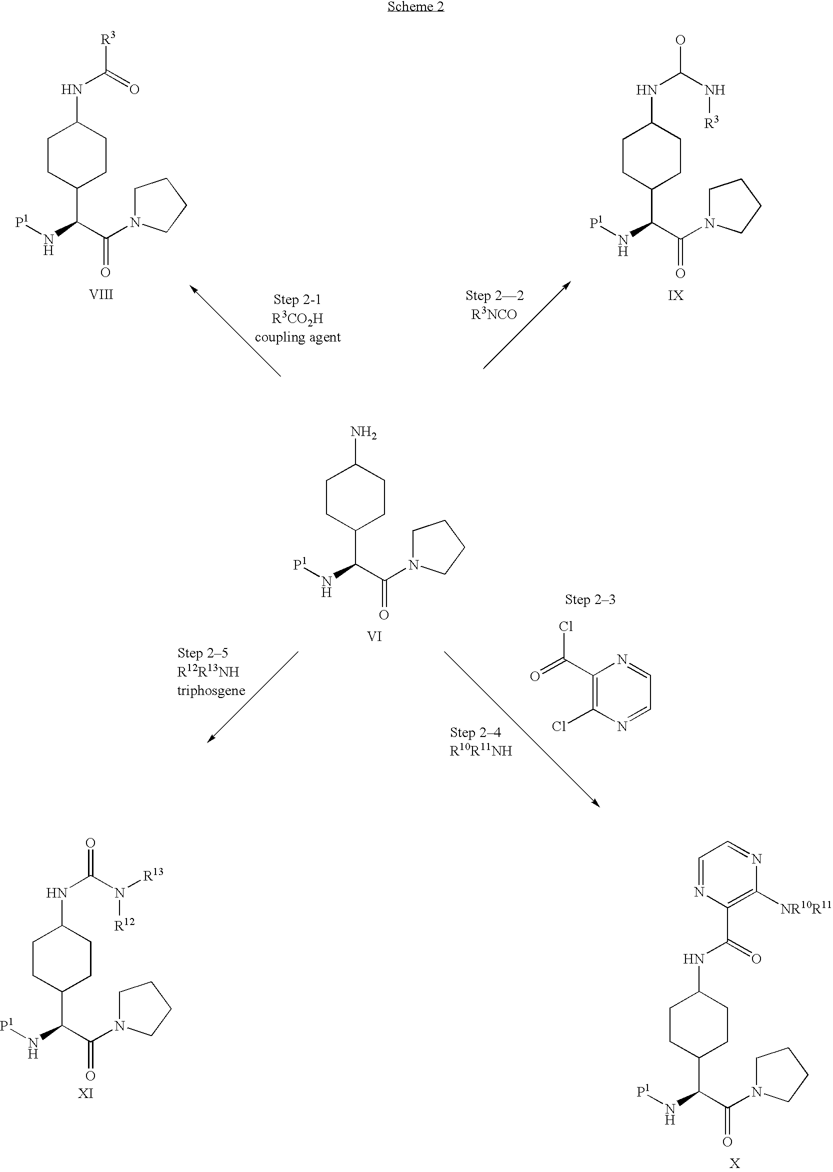

In step 2-3, the amine of formula VI is reacted with a halogenated heterocyclic acid chloride such as, for example, 2-chloro-3-pyrazinecarbonyl chloride, in an inert solvent (e.g. dichloromethane, THF), in the presence of a suitable base (e.g. triethylamine, pyridine). The reaction is conducted at a suitable temperature, such as 0-80° C., preferably at room temperature, for a suitable time, such as 1 to 24 hours, for example 16 hours.

In step 2-4, the resulting compound, for example 3-chloro-pyrazine-2-carboxamide, is dissolved in an inert solvent (e.g. dichloromethane, DMF) and treated at suitable temperature, such as 0-80° C., for a suitable time, such as 1 to 24 hours with an amine R⁷R⁸NH, wherein R⁷ and R⁸ are as defined above, in the presence of a suitable base (e.g. triethylamine, diisopropylethylamine), to yield a compound of Formula X. It will be clear to one skilled in the art that other halogenated heterocyclic acid chlorides (e.g. 2-chloro-3-pyridinecarbonyl chloride) can be used to give analogous derivatives.

Step 2-5 comprises reacting a compound of formula VI in a suitable solvent with an amine R¹²R¹³NH, where R¹² and R¹³ are linked together to form a 3- to 7-membered ring, optionally substituted with one to three hydroxy, aminocarbonyl, C₁₋₆alkylaminocarbonyl, cyano, phenyl-C₁₋₆alkylenylamino, benzylidene, benzyloxy-C₁₋₆alkylenyl, benzyloxycarbonyl or C₁₋₆alkoxycarbonyl, in the presence of a suitable base, such as a phosgene, diphosgene or triphosgene and a base (e.g. pyridine). Suitable solvents include dichloromethane and acetonitrile. The reaction is conducted at a suitable temperature, such as 0-25° C., preferably at room temperature, for a suitable time, such as 1 to 72 hours, for example 65 hours. The product of step 2-5 is a trisubstituted urea of Formula XI.

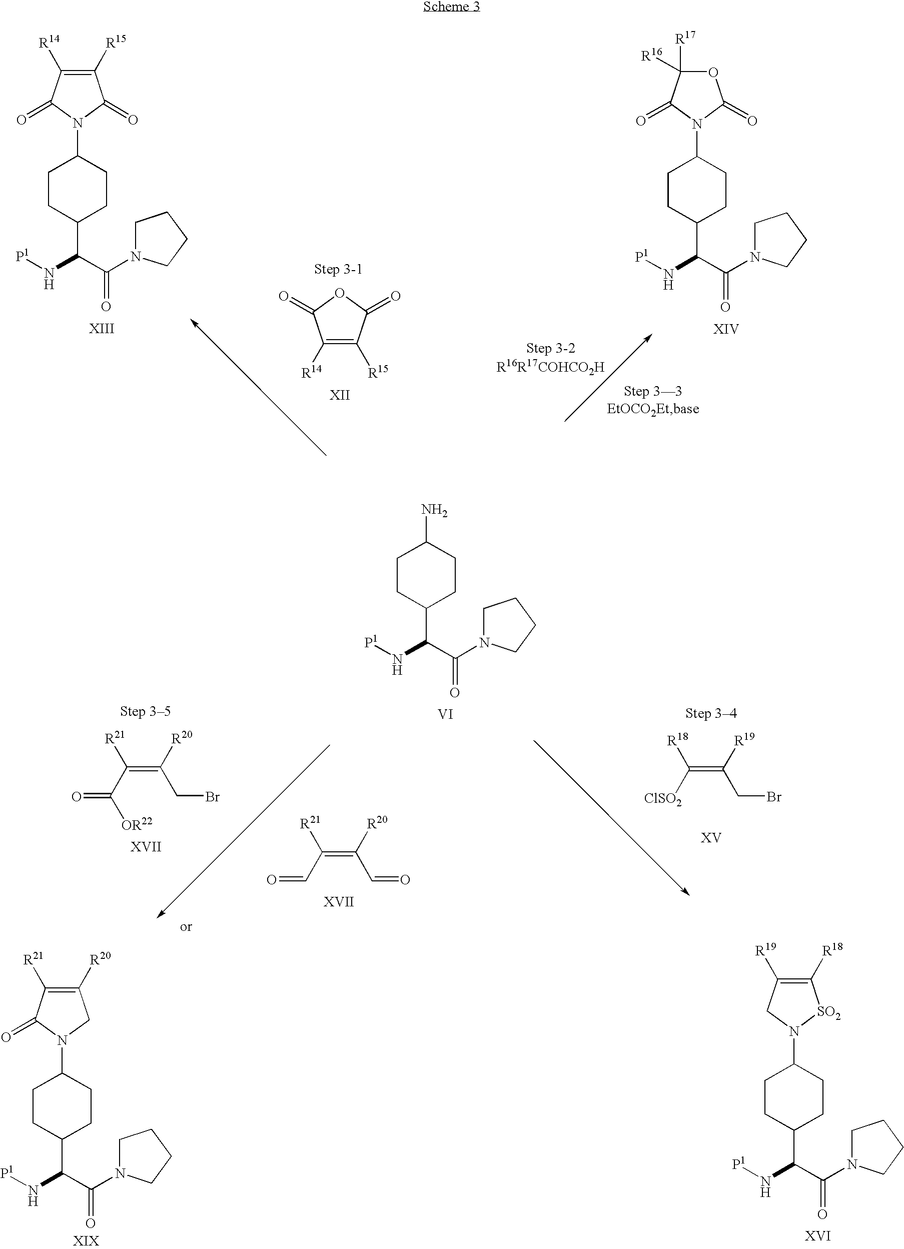

In Scheme 3, Step 3-1 comprises dissolving a compound of formula VI with an anhydride of formula XII, wherein R¹⁴ and R¹⁵ are joined together to form a heteroaromatic ring such as pyridine or pyrazine. Typically the two components are dissolved in an inert solvent (e.g. THF, DMF) and heated to a suitable temperature, such as 50-100° C., preferably 65° C., for a suitable time, for example, until the reaction is complete, typically within 2-24 hours. The product is a compound of Formula XIII.

In step 3-2 the amine of formula VI is reacted with a carboxylic acid of formula R¹⁶R¹⁷(OH)CCOOH, where R¹⁶ and R¹⁷ are independently hydrogen, C₁₋₆alkyl, or phenyl groups in the presence of a coupling agent, wherein suitable coupling agents and reaction conditions are as described above for step 1-3.

In step 3-3, the product of step 3-2 is heated to a suitable temperature, such as 50-150° C., with dimethyl or diethyl carbonate in the presence of a suitable base (e.g. sodium ethoxide) to yield an oxazolidinedione of formula XIV. The carbonate is typically used as the solvent. The reaction is conducted for a suitable time, such as 1 to 24 hours, for example 16 hours.

Step 3-4 comprises reacting a compound of formula VI with a bromosulfonyl chloride of formula XV, where R¹⁸ and R¹⁹ are joined together to form an aromatic carbocyclic or heterocyclic ring such as phenyl, pyridine, or pyrazine, in an inert solvent (e.g. THF, DMF) in the presence of a suitable base (e.g. potassium carbonate, triethylamine, pyridine) to provide a compound of Formula XVI. The reaction proceeds for a suitable time, such as ten minutes to 24 hours, at a suitable temperature, such as 0-50° C., preferably at room temperature.

Step 3-5 comprises reacting formula VI with a bromoester of formula XVII where R²⁰ and R²¹ are joined together to form a heteroaromatic ring such as pyridine or pyrazine, wherein R²² is C₃₋₆alkyl or benzyl, under suitable conditions analogous to those of step 3-4. Alternatively the compound of formula VI may be combined with a dialdehyde XVIII in an inert solvent (e.g. xylenes, toluene), and heated to a suitable temperature, such as 50-200° C., preferably 140° C., for a suitable time, such as 1 to 24 hours, preferably 16 hours, to yield the product of Formula XIX.

An alternative sequence to that shown in Scheme 1 is shown in Scheme 4, shown below, whereby the carboxylic acid function of IV is protected as an ester XX, where R²³ is C₃₋₆alkyl or benzyl. This is accomplished by alkylation of IV with an alkyl halide such as methyl iodide in the presence of a suitable base (e.g. potassium carbonate) in an inert solvent (e.g. DMF, THF) at suitable temperature, such as 0-50° C., preferably room temperature, for a suitable time, such as 1 to 24 hours, preferably 4 hours, or by an acid catalyzed reaction with an alcohol such as methanol. In the acid catalyzed reaction, the alcohol is typically used as the solvent and the reaction is carried out at suitable temperature, such as 20-80° C., for a suitable time, such as for 1 to 24 hours. It will be recognized by those skilled in the art that the conditions are chosen so as to be compatible with the presence of the protecting group P¹.

The ester XX is then subjected to a series of steps 4-2 to 4-5, which are analogous to the conditions described in steps 1-4 to 1-7 of Scheme 1 above, wherein P¹, R², R³, R⁹, and M are also as defined in Scheme 1. The product of step 4-5, formula XXII, is cleaved by saponification (step 4-6) to yield a corresponding carboxylic acid. This is typically accomplished by dissolving XXII in a water-miscible solvent (e.g. methanol, ethanol) and water in the presence of a suitable base (e.g. lithium hydroxide, sodium hydroxide) at suitable temperature, such as 0-100° C., preferably room temperature, for a suitable time, such as 1 to 24 hours, for example 16 hours.

In step 4-7, the product of step 4-6 is coupled under conditions as previously described in step 1-3. In step 4-8, the product of step 4-7 is subjected to deprotection under conditions as previously described for step 1-8 to yield a compound of Formula (I).

Practitioners will appreciate that a compound of Formula (I) where X is —CN, may also be prepared by Scheme 4, provided that an additional dehydration step under conditions analogous to those previously described for step 1-9 is included in the sequence. For example, after the coupling step of 4-7, the product may be subjected to dehydration conditions as described in step 1-9, and the product thereafter subjected to deprotection under conditions as previously described in step 1-8.

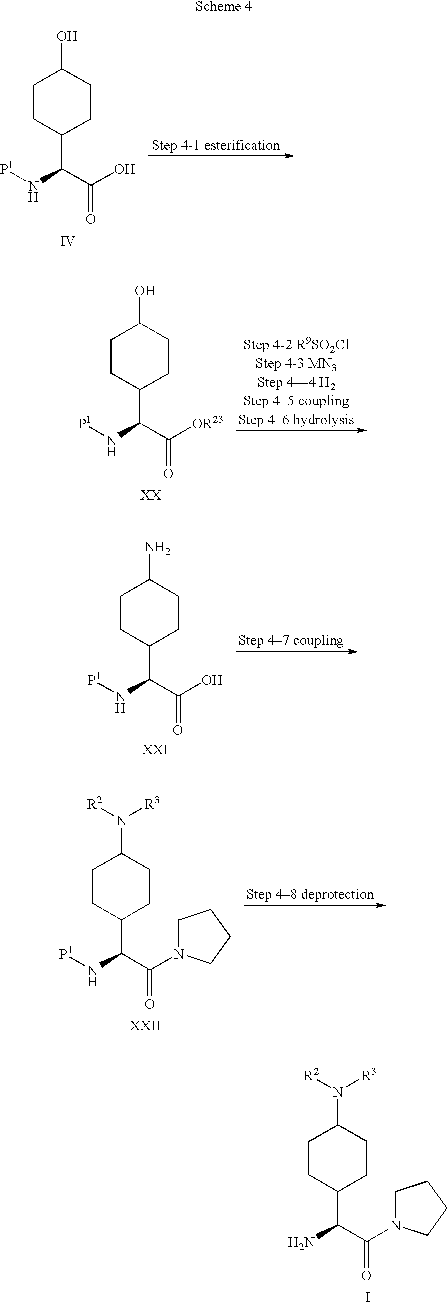

Practitioners will appreciate that the protected starting amino acid, Formula (I)I, which is depicted as having the L configuration in Scheme 1, may be a mixture of D and L isomers. Consequently, the compounds of Formula (I) may exist as DL mixtures and theses mixtures are within the scope of this invention.

Preferably, a pharmaceutical composition of the present invention comprises a therapeutically effective amount of a compound of Formula (IA), or a prodrug thereof, or a pharmaceutically acceptable salt of the compound or prodrug, or a solvate of the compound, prodrug or salt, and a pharmaceutically acceptable carrier, vehicle, diluent or excipient.

More preferably, a pharmaceutical composition of the present invention comprises a therapeutically effective amount of a compound of Formula (IB), or a prodrug thereof, or a pharmaceutically acceptable salt of the compound or prodrug, or a solvate of the compound, prodrug or salt, and a pharmaceutically acceptable carrier, vehicle, diluent or excipient.

Even more preferably, a pharmaceutical composition of the present invention comprises a therapeutically effective amount of the compound (S)-3-amino-pyrazine-2-carboxylic acid [trans-4-(1-amino-2-oxo-2-thiazolidin-3-yl-ethyl)-cyclohexyl]-amide, or a prodrug thereof, or a pharmaceutically acceptable salt of said compound or prodrug, or a solvate of said compound, prodrug or salt; and a pharmaceutically acceptable carrier, vehicle, diluent or excipient.

The pharmaceutical compositions formed by combining the compounds of this invention and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like.

Thus, for purposes of oral administration, tablets containing various excipients such as sodium citrate, calcium carbonate and/or calcium phosphate, may be employed along with various disintegrants such as starch, alginic acid and/or certain complex silicates, together with binding agents such as polyvinylpyrrolidone, sucrose, gelatin and/or acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the active pharmaceutical agent therein may be combined with various sweetening or flavoring agents, coloring matter or dyes and, if desired, emulsifying or suspending agents, together with diluents such as water, ethanol, propylene glycol, glycerin and/or combinations thereof.

For parenteral administration, solutions of the compounds or compositions of this invention in sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

For intranasal administration or administration by inhalation, the compounds or compositions of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of a compound of this invention. Capsules and cartridges (made, for example, from gelatin) for use in an inhaler or insufflator may be formulated containing a powder mix of a compound or compounds of the invention and a suitable powder base such as lactose or starch.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

In another aspect, the invention is directed to a pharmaceutical composition, which comprises a therapeutically effective amount of a first compound of Formula (I), a prodrug thereof or a pharmaceutically acceptable salt of the compound or the prodrug; a second compound that is an antidiabetic agent selected from insulin and insulin analogs; insulinotropin; biguanides; α₂-antagonists and imidazolines; glitazones; aldose reductase inhibitors; glycogen phosphorylase inhibitors; sorbitol dehydrogenase inhibitors; fatty acid oxidation inhibitors; α-glucosidase inhibitors; β-agonists; phosphodiesterase inhibitors; lipid-lowering agents; antiobesity agents; vanadate and vanadium complexes and peroxovanadium complexes; amylin antagonists; glucagon antagonists; growth hormone secretagogues; gluconeogenesis inhibitors; somatostatin analogs; antilipolytic agents; a prodrug of the antidiabetic agents, or a pharmaceutically acceptable salt of the antidiabetic agents and the prodrugs.

In another aspect, the invention is directed to a kit comprising: a first dosage form comprising a compound of Formula (I), or a prodrug thereof, or a pharmaceutically acceptable salt of the compound or prodrug, or a solvate of the compound, prodrug or salt; and a second dosage form comprising an antidiabetic agent selected from insulin and insulin analogs; insulinotropin; biguanides; α₂-antagonists and imidazolines; glitazones; aldose reductase inhibitors; glycogen phosphorylase inhibitors; sorbitol dehydrogenase inhibitors; fatty acid oxidation inhibitors; α-glucosidase inhibitors; β-agonists; phosphodiesterase inhibitors; lipid-lowering agents; antiobesity agents; vanadate and vanadium complexes and peroxovanadium complexes; amylin antagonists; glucagon antagonists; growth hormone secretagogues; gluconeogenesis inhibitors; somatostatin analogs; antilipolytic agents; prodrugs of the antidiabetic agents, or a pharmaceutically acceptable salts of the antidiabetic agents and the prodrug; and a container for containing said first dosage (a) and said second dosage (b). In a preferred embodiment of the kit, both the first and the second dosage forms independently comprise a pharmaceutically acceptable carrier or diluent.

In another aspect, the invention is directed to a therapeutic method of inhibiting dipeptidyl peptidase-IV comprising administering to a mammal in need of such treatment a therapeutically effective amount of a compound of Formula (I), or a prodrug thereof, or a pharmaceutically acceptable salt of the compound or of the prodrug, or a solvate of the compound, prodrug or salt; either alone or in combination with an antidiabetic agent as described above.

In another aspect, the invention is directed to a method of treating a condition mediated by dipeptidyl peptidase-IV inhibition comprising administering to a mammal in need of such treatment a therapeutically effective amount of a compound of Formula (I), or a prodrug thereof, or a pharmaceutically acceptable salt of the compound or of the prodrug, or a solvate of the compound, prodrug or salt; either alone or in combination with an antidiabetic agent as described above.

In one embodiment, the condition treated is Type 2 diabetes, Type 1 diabetes, impaired glucose tolerance, hyperglycemia, metabolic syndrome (syndrome X and/or insulin resistance syndrome), glucosuria, metabolic acidosis, arthritis, cataracts, diabetic neuropathy, diabetic nephropathy, diabetic retinopathy, diabetic cardiomyopathy, obesity, conditions exacerbated by obesity, hypertension, hyperlipidemia, atherosclerosis, osteoporosis, osteopenia, frailty, bone loss, bone fracture, acute coronary syndrome, short stature due to growth hormone deficiency, infertility due to polycystic ovary syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome; short bowel syndrome; and the prevention of disease progression in Type 2 diabetes.

In a preferred embodiment, the condition treated is Type 2 diabetes.

In another aspect, the invention is directed to a method of identifying an insulin secretagogue agent for diabetes, comprising: administering an agent of Formula (I) to a fasted, diabetic KK/H1J symptomatic mouse; and assessing a response in the mouse to a subsequent oral glucose challenge, wherein, if said mouse demonstrates an improvement in the symptoms, said agent is identified as a treatment for Type 2 diabetes, Type 1 diabetes, impaired glucose tolerance, hyperglycemia, metabolic syndrome (syndrome X and/or insulin resistance syndrome), glucosuria, metabolic acidosis, arthritis, cataracts, diabetic neuropathy, diabetic nephropathy, diabetic retinopathy, diabetic cardiomyopathy, obesity, conditions exacerbated by obesity, hypertension, hyperlipidemia, atherosclerosis, osteoporosis, osteopenia, frailty, bone loss, bone fracture, acute coronary syndrome, short stature due to growth hormone deficiency, infertility due to polycystic ovary syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome; short bowel syndrome, and to prevent disease progression in Type 2 diabetes.

The present invention also relates to therapeutic methods for treating or preventing the above described conditions in a mammal, including a human, wherein a compound of Formula (I) of this invention is administered as part of an appropriate dosage regimen designed to obtain the benefits of the therapy. The appropriate dosage regimen, the amount of each dose administered and the intervals between doses of the compound will depend upon the compound of Formula (I) of this invention being used, the type of pharmaceutical compositions being used, the characteristics of the subject being treated and the severity of the conditions.

In general, an effective dosage for the compounds of the present invention is in the range of 0.01 mg/kg/day to 30 mg/kg/day, preferably 0.01 mg/kg/day to 5 mg/kg/day of active compound in single or divided doses. Some variation in dosage will necessarily occur, however, depending on the condition of the subject being treated. The individual responsible for dosing will, in any event, determine the appropriate dose for the individual subject. Practitioners will appreciate that “kg” refers to the weight of the patient measured in kilograms.

The compounds or compositions of this invention may be administered in single (e.g., once daily) or multiple doses or via constant infusion. The compounds of this invention may also be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents.

The compounds or compositions of the present invention may be administered to a subject in need of treatment by a variety of conventional routes of administration, including orally and parenterally, (e.g., intravenously, subcutaneously or intramedullary). Further, the pharmaceutical compositions of this invention may be administered intranasally, as a suppository, or using a “flash” formulation, i.e., allowing the medication to dissolve in the mouth without the need to use water.

### EXEMPLIFICATION

Unless noted otherwise, all reactants were obtained commercially.

Flash chromatography was performed according to the method described by W. C. Still et al. in J. Org. Chem. 1978, 43, 2923.

Hydrogenations were performed in a Parr (Moline, Ill.) 3911 shaker type hydrogenation apparatus (hereafter referred to as a Parr hydrogenator) at the pressures indicated. NMR chemical shifts are given in parts per million downfield from tetramethylsilane (for proton) or fluorotrichloromethane (for fluorine). Spectra were recorded on a Varian (Palo Alto, Calif.) Unity-400 MHz spectrometer. Mass spectra were recorded on a Waters (Milford, Mass.) Micromass Platform II spectrometer.

The Examples set forth herein below are for illustrative purposes only. The compositions, methods, and various parameters reflected therein are intended only to exemplify various aspects and embodiments of the invention, and are not intended to limit the scope of the claimed invention in any way.

The compounds and intermediates of the present invention may be named according to either the IUPAC (International Union for Pure and Applied Chemistry) or CAS (Chemical Abstracts Service, Columbus, Ohio) nomenclature systems.

### Examples 1-7

The compounds of Examples 1-7 were prepared using (S)-[1-(cis-4-amino-cyclohexyl)-2-oxo-2-pyrrolidin-1-yl-ethyl]-carbamic acid tert-butyl ester, shown below, which was synthesized as follows.

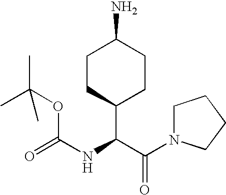

### Step 1: (S)-tert-Butoxycarbonylamino-(trans-4-hydroxy-cyclohexyl)-acetic acid

A mixture of 4-hydroxy-L-phenylglycine (15 g, 90 mmol) and Raney Nickel (30 g) in 3 N sodium hydroxide (30 mL) and water (220 mL) was hydrogenated at 40 psi and 55° C. overnight. The mixture was cooled to room temperature and filtered over diatomaceous earth, then concentrated to about half its volume. The solution was diluted with water (180 mL) and dioxane (120 mL) and treated with triethylamine (22.6 mL, 162 mmol) and di-tert-butyl dicarbonate (23.6 g, 108 mmol). The reaction mixture was concentrated to about half its volume, cooled to 0° C., acidified to pH 2-3 with 10% potassium bisulfate then extracted with ethyl acetate (3×). The combined extracts were washed with brine, dried over magnesium sulfate and concentrated to dryness, leaving a white foam (21 g, 85%).

### Step 2: (S)-[1-(trans-4-Hydroxy-cyclohexyl)-2-oxo-2-pyrrolidin-1-yl-ethyl]-carbamic acid tert-butyl ester

To a solution of (S)-tert-butoxycarbonylamino-(trans-4-hydroxy-cyclohexyl)-acetic acid (21 g, 77 mmol) in DMF (240 mL) was added pyrrolidine (7.7 mL, 92 mmol), triethylamine (24 mL, 1691 mmol) and benzotriazol-1-yloxytris(dimethylamino)phosphonium hexafluorophosphate (BOP) (38 g, 85 mmol). The mixture was stirred overnight, poured into water and extracted with dichloromethane (2×). The combined extracts were washed with 2 N hydrochloric acid, water, saturated sodium bicarbonate and brine, dried over magnesium sulfate and concentrated to dryness, leaving a white foam (25 g, 99%).

### Step 3: (S)-[1-(cis-4-Azido-cyclohexyl)-2-oxo-2-pyrrolidin-1-yl-ethyl]-carbamic acid tert-butyl ester

A solution of (S)-[1-(trans-4-hydroxy-cyclohexyl)-2-oxo-2-pyrrolidin-1-yl-ethyl]-carbamic acid tert-butyl ester (15.5 g, 47.5 mmol), in THF (60 mL) was cooled to 0° C. and treated with triphenylphosphine (13.8 g, 53 mmol), diethyl azodicarboxylate (8.05 mL, 51 mmol) and diphenylphosphoryl azide (11.3 mL, 53 mmol). The mixture was slowly warmed to room temperature over 18 hours and concentrated to dryness. The product was isolated by flash-chromatography (hexane/ethyl acetate, 3:1, then 2:1, then 1:1) as an oil (3.96 g, 27%).

### Step 4: (S)-[1-(cis-4-Amino-cyclohexyl)-2-oxo-2-pyrrolidin-1-yl-ethyl]-carbamic acid tert-butyl ester

A solution of (S)-[1-(cis-4-azido-cyclohexyl)-2-oxo-2-pyrrolidin-1-yl-ethyl]-carbamic acid tert-butyl ester (3.9 g, 11.1 mmol) in ethanol (50 mL) containing 10% palladium on carbon (400 mg) was treated with hydrogen in a Parr hydrogenator at 45 psi overnight. The reaction mixture was filtered through diatomaceous earth. The filtrate was concentrated to dryness, leaving an oil (3.8 g, 100%).

### Example 1

The hydrochloride salt of N-{[cis-4-((1S)-1-amino-2-oxo-2-pyrrolidin-1-yl-ethyl)-cyclohexylcarbamoyl]-methyl}-benzamide, shown below, was prepared as follows.

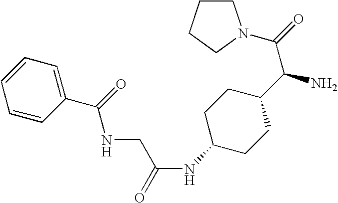

### Step 1: (S)-{1-[trans-4-(2-Benzoylamino-acetylamino)-cyclohexyl]-2-oxo-2-pyrrolidin-1-yl-ethyl}-carbamic acid tert-butyl ester

1-(3-Dimethylaminopropyl)-3-ethylcarbodiimide hydrochloride (71 mg, 0.37 mmol) was added to a solution of [(S)-1-(cis-4-amino-cyclohexyl)-2-oxo-2-pyrrolidin-1-yl-ethyl]-carbamic acid tert-butyl ester, (100 mg, 0.31 mmol), hippuric acid (66 mg, 0.37 mmol) and hydroxybenzotriazole (50 mg, 0.37 mmol) in dichloromethane (5 mL). The mixture was stirred overnight at room temperature, then concentrated and the residue was diluted with ethyl acetate, washed with 2 N sodium hydroxide, water and brine, dried over magnesium sulfate and concentrated. The residue was purified by flash-chromatography (ethyl acetate) and the product was obtained as a white solid (39 mg, 26%).

### Step 2: N-{[cis-4-((1S)-1-Amino-2-oxo-2-pyrrolidin-1-yl-ethyl)-cyclohexylcarbamoyl]-methyl}-benzamide hydrochloride

The product was dissolved in ethyl acetate (3 mL), the solution was cooled to 0° C., saturated with hydrogen chloride, and stirred for 20 min at room temperature. The solvent was evaporated and the resulting off-white solid was dried under vacuum (23 mg, 80%). MS m/z 387 (MH⁺).

### Example 2

The hydrochloride salt of {[cis-4-((1S)-1-amino-2-oxo-2-pyrrolidin-1-yl-ethyl)-cyclohexylcarbamoyl]-methyl}-carbamic acid benzyl ester, shown below, was prepared by the method of Example 1 using (S)-[1-(cis-4-amino-cyclohexyl)-2-oxo-2-pyrrolidin-1-yl-ethyl]-carbamic acid tert-butyl ester and carbobenzyloxy-glycine. MS m/z 417 (MH⁺).

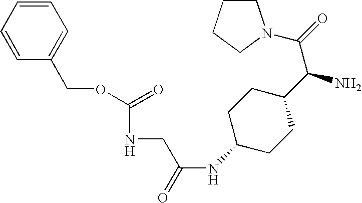

### Example 3

The hydrochloride salt of (2S)-2-[cis-4-(1-(1S)-amino-2-oxo-2-pyrrolidin-1-yl-ethyl)-cyclohexylcarbamoyl]-pyrrolidine-1-carboxylic acid benzyl ester, shown below, was prepared by the method of Example 1 using [(S)-1-(cis-4-amino-cyclohexyl)-2-oxo-2-pyrrolidin-1-yl-ethyl]-carbamic acid tert-butyl ester and N-carbobenzyloxy-L-proline. MS m/z 457 (MH⁺).

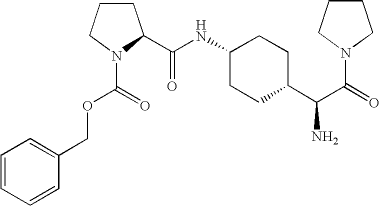

### Example 4

The hydrochloride salt of (4S)-4-{2-[cis-4-((1S)-1-amino-2-oxo-2-pyrrolidin-1-yl-ethyl)-cyclohexylcarbamoyl]-ethyl}-5-oxo-oxazolidine-3-carboxylic acid benzyl ester hydrochloride, shown below, was prepared by the method of Example 1 using [(S)-1-(cis-4-amino-cyclohexyl)-2-oxo-2-pyrrolidin-1-yl-ethyl]-carbamic acid S-benzyloxycarbonyl-5-oxo-4-oxazolidinepropionic acid. MS m/z 501 (MH⁺).

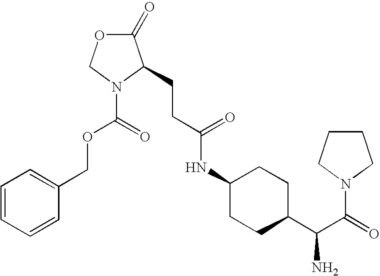

### Example 5

The hydrochloride salt of (4R)-4-[cis-4-((1S)-1-amino-2-oxo-2-pyrrolidin-1-yl-ethyl)-cyclohexylcarbamoyl]-oxazolidine-3-carboxylic acid benzyl ester, shown below, was prepared by the method of Example 1 using [(S)-1-(cis-4-amino-cyclohexyl)-2-oxo-2-pyrrolidin-1-yl-ethyl]-carbamic acid tert-butyl ester and (R)-Carbobenzyloxy-oxaproline. MS m/z 459 (MH⁺).

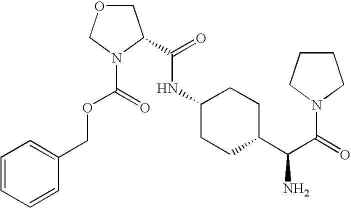

### Example 6

The hydrochloride salt of (4S)-4-[cis-4-((1S)-1-amino-2-oxo-2-pyrrolidin-1-yl-ethyl)-cyclohexylcarbamoyl]-oxazolidine-3-carboxylic acid benzyl ester, shown below, was prepared by the method of Example 1 using [(S)-1-(cis-4-amino-cyclohexyl)-2-oxo-2-pyrrolidin-1-yl-ethyl]-carbamic acid tert-butyl ester and (S)-Carbobenzyloxy-oxaproline. MS m/z 459 (MH⁺).

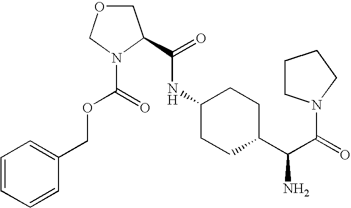

### Example 7

The hydrochloride salt of (5S)-5-[cis-4-((1S)-1-amino-2-oxo-2-pyrrolidin-1-yl-ethyl)-cyclohexylcarbamoyl]-2-oxo-imidazolidine-1-carboxylic acid benzyl ester, shown below, was prepared by the method of Example 1 using [(S)-1-(cis-4-amino-cyclohexyl)-2-oxo-2-pyrrolidin-1-yl-ethyl]-carbamic acid tert-butyl ester and (S)-2-oxo-1,5-imidazolinedicarboxylic acid 1-benzyl ester. MS m/z 472 (MH⁺).

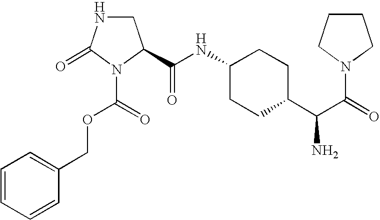

### Examples 8-28

The compounds of Examples 8-28 were prepared using (S)-[1-(trans-4-amino-cyclohexyl)-2-oxo-2-pyrrolidin-1-yl-ethyl]-carbamic acid tert-butyl ester, shown below, which was synthesized as follows.

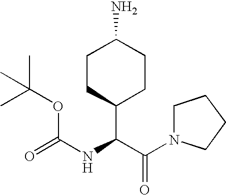

### Step 1: (S)-tert-Butoxycarbonylamino-(cis-4-hydroxy-cyclohexyl)-acetic acid

Boc-(L)-Phenylglycine (24 g, 90 mmol) was dissolved in ethanol (100 mL), 5% rhodium on carbon (3.5 g) was added and the mixture was hydrogenated at 40 psi for 3 days. The mixture was filtered over diatomaceous earth, then concentrated to a foam (21.6 g, 88%).

### Step 2: (S)-[1-(cis-4-Hydroxy-cyclohexyl)-2-oxo-2-pyrrolidin-1-yl-ethyl]-carbamic acid tert-butyl ester

(S)-tert-Butoxycarbonylamino-(cis-4-hydroxy-cyclohexyl)-acetic acid was coupled with pyrrolidine as described in Step 2 of the method for preparing [(S)-1-(cis-4-amino-cyclohexyl)-2-oxo-2-pyrrolidin-1-yl-ethyl]-carbamic acid tert-butyl ester.

### Step 3: (S)-Methanesulfonic acid cis-4-(1-tert-Butoxycarbonylamino-2-oxo-2-pyrrolidin-1-yl-ethyl)-cyclohexyl ester

To a solution of (S)-[1-(cis-4-hydroxy-cyclohexyl)-2-oxo-2-pyrrolidin-1-yl-ethyl]-carbamic acid tert-butyl ester (4.05 g, 12.4 mmol) and diisopropylethylamine (4.3 mL, 25 mmol) in dichloromethane (20 mL) was added drop wise at 0° C. methanesulfonyl chloride (1.44 mL, 19 mmol). The mixture was stirred at 0° C. for 10 minute then diluted with ethyl acetate, washed with saturated sodium bicarbonate (2×), water and brine, dried over magnesium sulfate and concentrated to a solid (5.02 g, 100%).

### Step 4: (S)-[1-trans-(4-Azido-cyclohexyl)-2-oxo-2-pyrrolidin-1-yl-ethyl]-carbamic acid tert-butyl ester

To a solution of (S)-methanesulfonic acid cis-4-(1-tert-butoxycarbonylamino-2-oxo-2-pyrrolidin-1-yl-ethyl)-cyclohexyl ester (5.0 g, 12 mmol) in DMF (30 mL) was added lithium azide (1.81 g, 37 mmol). The mixture was heated to 65° C. overnight, cooled, diluted with ethyl acetate, washed with water, 4% magnesium sulfate solution and brine, dried over magnesium sulfate and concentrated to dryness, leaving a yellow oil (3.8 g, 87%).

### Step 5: (S)-[1-(trans-4-Amino-cyclohexyl)-2-oxo-2-pyrrolidin-1-yl-ethyl]-carbamic acid tert-butyl ester

(S)-[1-trans-(4-Azido-cyclohexyl)-2-oxo-2-pyrrolidin-1-yl-ethyl]-carbamic acid tert-butyl ester was hydrogenated as in Step 4 of the method for preparing [(S)-1-(cis-4-amino-cyclohexyl)-2-oxo-2-pyrrolidin-1-yl-ethyl]-carbamic acid tert-butyl ester.

### Example 8

The hydrochloride salt of (4R)-4-[trans-4-((1S)-1-amino-2-oxo-2-pyrrolidin-1-yl-ethyl)-cyclohexylcarbamoyl]-oxazolidine-3-carboxylic acid benzyl ester, shown below, was prepared by the method of Example 1 using [(S)-1-(trans-4-amino-cyclohexyl)-2-oxo-2-pyrrolidin-1-yl-ethyl]-carbamic acid tert-butyl ester and (R)-Carbobenzyloxy-oxaproline. MS m/z 459 (MH⁺).

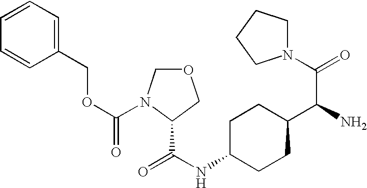

### Example 9

The hydrochloride salt of (5S)-5-[trans-4-((1S)-1-amino-2-oxo-2-pyrrolidin-1-yl-ethyl)-cyclohexylcarbamoyl]-2-oxo-imidazolidine-1-carboxylic acid benzyl ester, shown below, was prepared by the method of Example 1 using [(S)-1-(trans-4-amino-cyclohexyl)-2-oxo-2-pyrrolidin-1-yl-ethyl]-carbamic acid tert-butyl ester and (S)-2-oxo-1,5-imidazolinedicarboxylic acid 1-benzyl ester. MS m/z 472 (MH⁺).

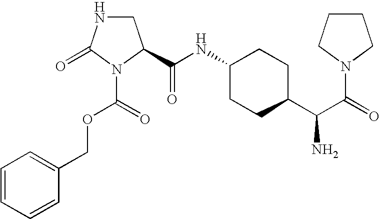

### Example 10

The hydrochloride salt of (4S)-4-{2-[trans-4-((1S-)-1-amino-2-oxo-2-pyrrolidin-1-yl-ethyl)-cyclohexylcarbamoyl]-ethyl}-5-oxo-oxazolidine-3-carboxylic acid benzyl ester, shown below, was prepared by the method of Example 1 using [(S)-1-(trans-4-amino-cyclohexyl)-2-oxo-2-pyrrolidin-1-yl-ethyl]-carbamic acid tert-butyl ester and (S)-3-Carbobenzyloxy-5-oxo-4-oxazolinepropionic acid. MS m/z 501 (MH⁺).

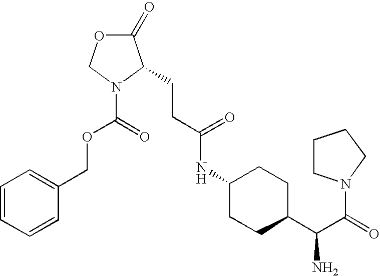

### Example 11

The hydrochloride salt of (4S)-4-[trans-4-((1S)-1-amino-2-oxo-2-pyrrolidin-1-yl-ethyl)-cyclohexylcarbamoyl]-oxazolidine-3-carboxylic acid benzyl ester, shown below, was prepared by the method of Example 1 using [(S)-1-(trans-4-amino-cyclohexyl)-2-oxo-2-pyrrolidin-1-yl-ethyl]-carbamic acid tert-butyl ester and (S)-Carbobenzyloxy-oxaproline. MS m/z 459 (MH⁺).

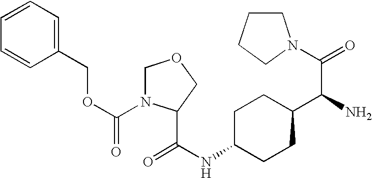

### Example 12

The hydrochloride salt of (2S,4R)-2-[trans-4-((1S)-1-amino-2-oxo-2-pyrrolidin-1-yl-ethyl)-cyclohexylcarbamoyl]-4-hydroxy-pyrrolidine-1-carboxylic acid benzyl ester, shown below, was prepared by the method of Example 1 using [(S)-1-(trans-4-amino-cyclohexyl)-2-oxo-2-pyrrolidin-1-yl-ethyl]-carbamic acid tert-butyl ester and N-carbobenzyloxy-L-hydroxyproline. MS m/z 473 (M⁺+1).

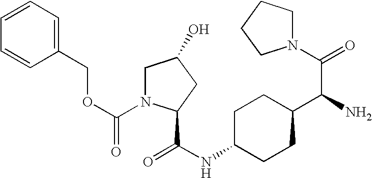

### Example 13

The hydrochloride salt of (4S)-4-{[trans-4-((1S)-1-amino-2-oxo-2-pyrrolidin-1-yl-ethyl)-cyclohexylcarbamoyl]-methyl}-5-oxo-oxazolidine-3-carboxylic acid benzyl ester, shown below, was prepared by the method of Example 1 using [(S)-1-(trans-4-amino-cyclohexyl)-2-oxo-2-pyrrolidin-1-yl-ethyl]-carbamic acid tert-butyl ester and (S)-benzyloxycarbonyl-5-oxo-4-oxazolidineacetic acid. MS m/z 487 (M⁺+1)

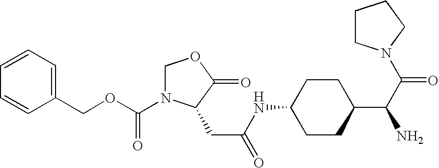

### Example 14

The hydrochloride salt of (2S)-2-[trans-4-(1-(1S)-amino-2-oxo-2-pyrrolidin-1-yl-ethyl)-cyclohexylcarbamoyl]-5-oxo-pyrrolidine-1-carboxylic acid benzyl ester, shown below, was prepared by the method of Example 1 using [(S)-1-(trans-4-amino-cyclohexyl)-2-oxo-2-pyrrolidin-1-yl-ethyl]-carbamic acid tert-butyl ester and benzyloxycarbonyl-D-pyroglutamic acid. MS m/z 471 (MH⁺).

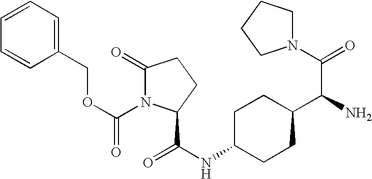

### Example 15

The hydrochloride salt of (2R)-2-[trans-4-(1-(S)-amino-2-oxo-2-pyrrolidin-1-yl-ethyl)-cyclohexylcarbamoyl]-5-oxo-pyrrolidine-1-carboxylic acid benzyl ester, shown below, was prepared by the method of Example 1 using [(S)-1-(trans-4-amino-cyclohexyl)-2-oxo-2-pyrrolidin-1-yl-ethyl]-carbamic acid tert-butyl ester and benzyloxycarbonyl-L-pyroglutamic acid. MS m/z 471 (MH⁺).

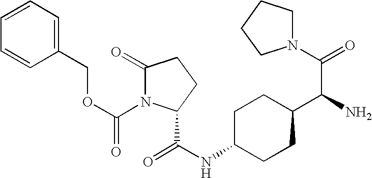

### Example 16

The hydrochloride salt of (4S)-3-benzyl-2-oxo-oxazolidine-4-carboxylic acid [trans-4-((1S)-1-Amino-2-oxo-2-pyrrolidin-1-yl-ethyl)-cyclohexyl]-amide, shown below, was prepared by the method of Example 1 using [(S)-1-(trans-4-amino-cyclohexyl)-2-oxo-2-pyrrolidin-1-yl-ethyl]-carbamic acid tert-butyl ester and (S)-3-benzyl-2-oxo-oxazolidine-4-carboxylic acid (Tetrahedron Asymm. 1994, 5, 161). MS m/z 420 (MH⁺).

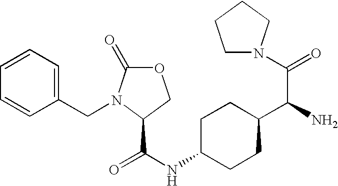

### Example 17

The hydrochloride salt of (S)-{1-[trans-4-(1-amino-2-oxo-2-pyrrolidin-1-yl-ethyl)-cyclohexylcarbamoyl]-1-methyl-ethyl)-methyl-carbamic acid benzyl ester, shown below, was prepared by the method of Example 1 using [(S)-1-(trans-4-amino-cyclohexyl)-2-oxo-2-pyrrolidin-1-yl-ethyl]-carbamic acid tert-butyl ester and N-Carbobenzyloxy-N,2-dimethylalanine. MS m/z 459 (MH⁺).

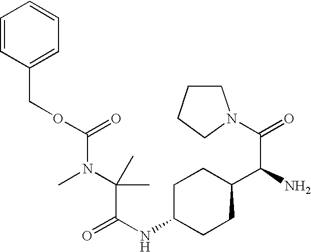

### Example 18

The hydrochloride salt of (S)-3-amino-pyrazine-2-carboxylic acid [trans-4-(1-Amino-2-oxo-2-pyrrolidin-1-yl-ethyl)-cyclohexyl]-amide, shown below, was prepared by the method of Example 1 using [(S)-1-(trans-4-amino-cyclohexyl)-2-oxo-2-pyrrolidin-1-yl-ethyl]-carbamic acid tert-butyl ester and 3-amino-2-pyrazinecarboxylic acid. MS m/z 459 (MH⁺).

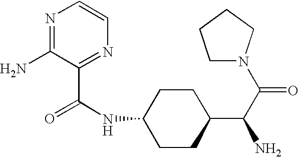

### Example 19

The hydrochloride salt of (S)-pyrazine-2,3-dicarboxylic acid amide [trans-4-(1-amino-2-oxo-2-pyrrolidin-1-yl-ethyl)-cyclohexyl]-amide, shown below, was prepared by the method of Example 1 using [(S)-1-(trans-4-amino-cyclohexyl)-2-oxo-2-pyrrolidin-1-yl-ethyl]-carbamic acid tert-butyl ester and pyrazine-2,3-dicarboxylic acid monoamide. ¹H NMR (CD₃OD, 400 MHz) 61.25-1.80 (m, 3H), 1.80-2.15 (m, 9H), 2.20-2.25 (m, 1H), 3.45-4.20 (m, 6H), 8.90-9.05 (m, 2H).

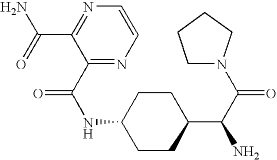

### Example 20

The hydrochloride salt of N-[trans-4-(1-(1S)-amino-2-oxo-2-pyrrolidin-1-yl-ethyl)-cyclohexyl]-2-(1-benzylidene-3-oxo-1,3-dihydro-isoindol-2-yl)-acetamide, shown below, was prepared by the method of Example 1 using [(S)-1-(trans-4-amino-cyclohexyl)-2-oxo-2-pyrrolidin-1-yl-ethyl]-carbamic acid tert-butyl ester and 3-benzylidene-1-oxo-2,3-dihydroisoindole-2-acetic acid. MS m/z 458 (MH⁺)

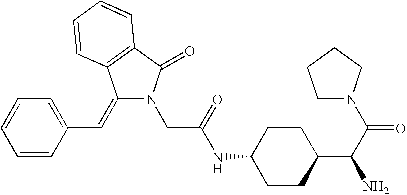

### Example 21

The hydrochloride salt of (1S,4R)-4-hydroxy-pyrrolidine-2-carboxylic acid [trans-4-((1S)-1-amino-2-oxo-2-pyrrolidin-1-yl-ethyl)-cyclohexyl]-amide, shown below, was prepared as follows.

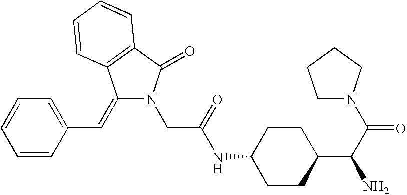

### Step 1: 2-[trans-4-((1S)-1-tert-Butoxycarbonylamino-2-oxo-2-pyrrolidin-1-yl-ethyl)-cyclohexylcarbamoyl]-(1S,4R)-4-hydroxy-pyrrolidine-1-carboxylic acid benzyl ester

(S)-[1-(trans-4-Amino-cyclohexyl)-2-oxo-2-pyrrolidin-1-yl-ethyl]-carbamic acid tert-butyl ester and N-carbobenzyloxy-L-hydroxyproline was coupled according to the procedure of Example 1.

### Step 2: (S)-(1-{trans-4-[((1S,4R)-4-Hydroxy-pyrrolidine-2-carbonyl)-amino]-cyclohexyl}-2-oxo-2-pyrrolidin-1-yl-ethyl)-carbamic acid tert-butyl ester

The product of step 1 (1.33 g, 2.3 mmol) was dissolved in ethanol (10 mL), 10% palladium on carbon (290 mg) was added and the mixture was treated with hydrogen at 35 psi for 16 hours. The solution was filtered through diatomaceous earth and the filtrate was concentrated to dryness, leaving a solid (832 mg, 82%).

### Step 3: (1S,4R)-4-Hydroxy-pyrrolidine-2-carboxylic acid [trans-4-(1-(S)-Amino-2-oxo-2-pyrrolidin-1-yl-ethyl)-cyclohexyl]-amide

The product of step 2 was treated with hydrogen chloride as described in Example 1. MS m/z 339 (MH⁺).

### Example 22

The hydrochloride salt of 6-[trans-4-((1S)-1-amino-2-oxo-2-pyrrolidin-1-yl-ethyl)-cyclohexyl]-pyrrolo[3,4-b]pyrazine-5,7-dione, shown below, was prepared as follows.

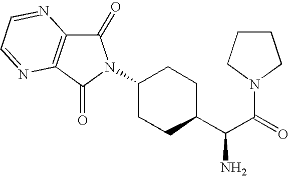

A solution of (S)-[1-(trans-4-amino-cyclohexyl)-2-oxo-2-pyrrolidin-1-yl-ethyl]-carbamic acid tert-butyl ester (176 mg, 0.5 mmol) and 2,3-pyrazinecarboxylic anhydride (75 mg, 0.5 mmol) in THF (5 mL) was stirred at reflux for 2 hours. The mixture was concentrated to dryness, treated with acetic anhydride (4 mL) and heated to reflux for 3 hours. The excess acetic anhydride was removed under high vacuum and the residue was partitioned between ethyl acetate and water. The aqueous layer was extracted with ethyl acetate and the combined organic phases were washed with water (3×) and brine, dried over magnesium sulfate and concentrated to dryness. The residue was triturated with ether and the light brown solid was isolated. This solid was dissolved in 4 N hydrogen chloride/dioxane (1 mL) and the solution was stirred at room temperature for 1 hour. Ether (2 mL) was added and the precipitate was collected and dried (17 mg, 8.6%). MS m/z 358 (MH⁺).

### Example 23

The hydrochloride salt of 2-[trans-4-((1S)-1-amino-2-oxo-2-pyrrolidin-1-yl-ethyl)-cyclohexyl]-pyrrolo[3,4-c]pyridine-1,3-dione, shown below, was prepared by the method of Example 22 using [(S)-1-(trans-4-amino-cyclohexyl)-2-oxo-2-pyrrolidin-1-yl-ethyl]-carbamic acid tert-butyl ester and 3,4-pyridinecarboxylic anhydride. MS m/z 357 (MH⁺).

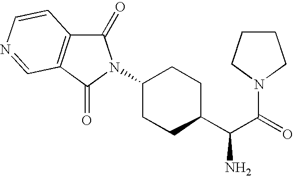

### Example 24

The hydrochloride salt of 6-[trans-4-((1S)-1-amino-2-oxo-2-pyrrolidin-1-yl-ethyl)-cyclohexyl]-pyrrolo[3,4-b]pyridine-5,7-dione, shown below, was prepared by the method of Example 22 using [(S)-1-(trans-4-amino-cyclohexyl)-2-oxo-2-pyrrolidin-1-yl-ethyl]-carbamic acid tert-butyl ester and 2,3-pyridinecarboxylic anhydride. MS m/z 357 (MH⁺).

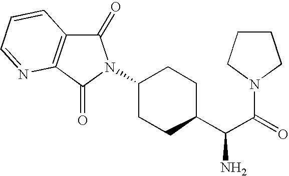

### Example 25

The hydrochloride salt of 2-benzyloxymethyl-pyrrolidine-1-carboxylic acid [trans-4-((1S)-1-Amino-2-oxo-2-pyrrolidin-1-yl-ethyl)-cyclohexyl]-amide, shown below, was prepared as follows.

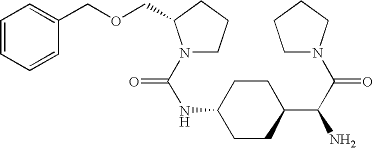

To a solution of triphosgene (37 mg, 0.125 mmol) in dichloromethane (2 mL), cooled to −10° C., was added a solution of (S)-[1-(trans-4-amino-cyclohexyl)-2-oxo-2-pyrrolidin-1-yl-ethyl]-carbamic acid tert-butyl ester (106 mg, 0.3 mmol). After 90 minutes a solution of (S)-(−)-2-[(benzyloxy)methyl]-pyrrolidine (J. Med. Chem. 1999, 42, 677) (205 mg, 0.9 mmol) and pyridine (0.14 mL, 1.75 mmol) in dichloromethane (2 mL) was added. The mixture was warmed to room temperature and stirred for 65 hours, then concentrated to dryness. The residue was taken up in 1:1 ether/ethyl acetate, washed with 1 N hydrochloric acid (2×) and brine, dried over magnesium sulfate and concentrated. The product was isolated by flash-chromatography (hexanes/ethyl acetate, 1:1 the ethyl acetate as a solid.

The solid was dissolved in 4 N hydrogen chloride in dioxane and the solution was stirred for 1 hour. The solvent was evaporated and the resulting solid was dried (12 mg, 9%). ¹H NMR (CD₃OD, 400 MHz) δ 0.90-2.01 (m, 17H), 3.41-3.55 (m, 8H), 3.64 (s, 3H), 3.97 (m, 2H), 4.48 (AB, J=11.2 Hz, 1H), 4.51 (AB, J=11.2 Hz, 1H), 7.27-7.34 (m, 5H).

### Example 26

The hydrochloride salt of (2S,4R)-4-hydroxy-pyrrolidine-1,2-dicarboxylic acid 2-{[trans-4-((1S)-1-Amino-2-oxo-2-pyrrolidin-1-yl-ethyl)-cyclohexyl]-amide}1-benzylamide, shown below, was prepared as follows.

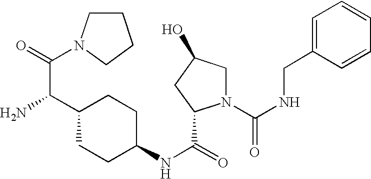

To a solution of (S)-(1-{trans-4-[((1S,4R)-4-hydroxy-pyrrolidine-2-carbonyl)-amino]-cyclohexyl}-2-oxo-2-pyrrolidin-1-yl-ethyl)-carbamic acid tert-butyl ester, Example 21, step 2, (88 mg, 0.2 mmol) in dichloromethane (3 mL) was added phenyl isocyanate (25 μL, 0.2 mmol). After 1 hour the solution was concentrated to dryness, leaving a white solid (115 mg, 100%). The tert-butoxycarbonyl group was removed as described in example 25 and the product was isolated as a colorless solid. MS m/z 472 (MH+).

### Example 27

The hydrochloride salt of 3-phenethylamino-pyrazine-2-carboxylic acid trans-4-((1S)-1-amino-2-oxo-2-pyrrolidin-1-yl-ethyl)-cyclohexyl]-amide, shown below, was prepared as follows.

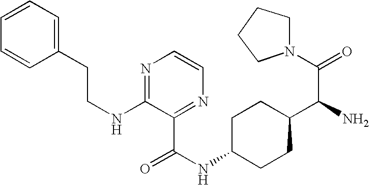

### Step 1: ((1S)-1-[trans-4-[(3-Chloro-pyrazine-2-carbonyl)-amino]-cyclohexyl}-2-oxo-2-pyrrolidin-1-yl-ethyl)-carbamic acid tert-butyl ester

A solution of (S)-[1-(trans-4-amino-cyclohexyl)-2-oxo-2-pyrrolidin-1-yl-ethyl]-carbamic acid tert-butyl ester (282 mg, 0.90 mmol), in dichloromethane (10 mL) was cooled to 0° C. and treated with a solution of 2-chloro-3-pyrazinecarbonyl chloride (Laduree, Heterocycles 1984, 22, 299-301) (159 mg, 0.90 mmol) in dichloromethane (5 mL). The mixture was warmed to room temperature, diluted with ethyl acetate, washed with 2 N hydrochloric acid, water and brine, dried over magnesium sulfate and concentrated to dryness (220 mg, 52%).

### Step 2: ((1S)-2-Oxo-1-{trans-4-[(3-phenethylamino-pyrazine-2-carbonyl)-amino]-cyclohexyl}-2-pyrrolidin-1-yl-ethyl)-carbamic acid tert-butyl ester

The above product (90 mg, 0.19 mmol) and triethylamine (30 μL, 0.21 mmol) was dissolved in dichloromethane (4 mL), cooled to 0° C. and treated with a solution of phenethylamine (40 μL, 0.29 mmol) in dichloromethane (1 mL). The reaction mixture was heated to 40° C. for 4 hours, cooled, diluted with ethyl acetate, washed with 2 N hydrochloric acid, water and brine, dried over magnesium sulfate and concentrated to dryness. The product was isolated by flash-chromatography as a solid (26 mg, 25%).

### Step 3: 3-Phenethylamino-pyrazine-2-carboxylic acid trans-4-((1S)-1-Amino-2-oxo-2-pyrrolidin-1-yl-ethyl)-cyclohexyl]-amide

The tert-butoxycarbonyl group was removed as described in Example 25 and the title product was isolated as a colorless solid. MS m/z 451 (MH+).

### Example 28

The hydrochloride salt of 3-[trans-4-((1S)-1-amino-2-oxo-2-pyrrolidin-1-yl-ethyl)-cyclohexyl]-(5S)-5-phenyl-oxazolidine-2,4-dione, shown below, was prepared as follows.

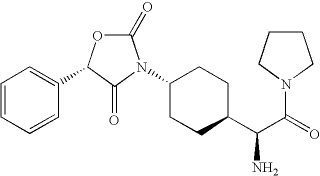

(S)-[1-(trans-4-Amino-cyclohexyl)-2-oxo-2-pyrrolidin-1-yl-ethyl]-carbamic acid tert-butyl ester and L-mandelic acid were coupled according to the Procedure of Example 1.

The product, ((1S)-{1-[trans-4-((2S)-2-hydroxy-2-phenyl-acetylamino)-cyclohexyl]-2-oxo-2-pyrrolidin-1-yl-ethyl}-carbamic acid tert-butyl ester (110 mg, 0.24 mmol) was mixed with diethyl carbonate (2 mL) and treated with a solution of sodium (1 mg) in ethanol (0.1 mL). The mixture was stirred at 135° C. for 16 hours then concentrated to dryness. The product was isolated by flash-chromatography (gradient of 1%, 2%, 3%, 5% methanol in dichloromethane) as a colorless solid (29.5 mg, 25%).

The solid was treated with HCl in dioxane as described in Example 25, giving the product as a solid. MS m/z 386 (MH⁺).

### Examples 29-33

The compounds of Examples 29-33 were prepared using ([(S)-1-(trans-4-amino-cyclohexyl)-2-(3,3-difluoro-pyrrolidin-1-yl)-2-oxo-ethyl]-carbamic acid tert-butyl ester, shown below, which was prepared from 2,3-pyridinecarboxylic anhydride. MS m/z 357 (MH⁺) using the method for preparing [(S)-1-(trans-4-amino-cyclohexyl)-2-oxo-2-pyrrolidin-1-yl-ethyl]-carbamic acid tert-butyl ester.

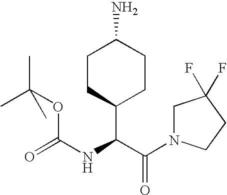

### Example 29

The hydrochloride salt of (5S)-5-{trans-4-[(1S)-1-amino-2-(3,3-difluoro-pyrrolidin-1-yl)-2-oxo-ethyl]-cyclohexylcarbamoyl}-2-oxo-imidazolidine-1-carboxylic acid benzyl ester, shown below, was prepared by the method of Example 1 using (S)-[1-(trans-4-amino-cyclohexyl)-2-(3,3-difluoro-pyrrolidin-1-yl)-2-oxo-ethyl]-carbamic acid tert-butyl ester and (S)-2-oxo-1,5-imidazoline carboxylic acid. MS m/z 508 (MH⁺).

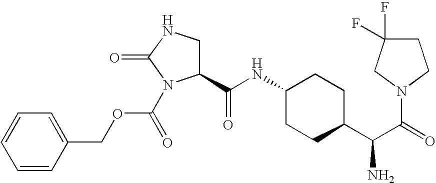

### Example 30

The hydrochloride salt of (S)-({trans-4-[1-amino-2-(3,3-difluoro-pyrrolidin-1-yl)-2-oxo-ethyl]-cyclohexylcarbamoyl}-methyl)-methyl-carbamic acid benzyl ester, shown below, was prepared as follows.

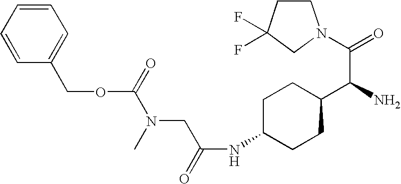

### Step 1: [(S)-1-{trans-4-[2-(Benzyloxycarbonyl-methyl-amino)-acetylamino]-cyclohexyl}-2-(3,3-difluoro-pyrrolidin-1-yl)-2-oxo-ethyl]-carbamic acid tert-butyl ester

1-(3-Dimethylaminopropyl)-3-ethylcarbodiimide hydrochloride (95 mg, 0.50 mmol) was added to a solution of (S)-[1-(trans-4-amino-cyclohexyl)-2-(3,3-difluoro-pyrrolidin-1-yl)-2-oxo-ethyl]-carbamic acid tert-butyl ester, (150 mg, 0.42 mmol), benzyl-N-(carboxymethyl)-N-methylcarbamate (111 mg, 0.50 mmol) and hydroxybenzotriazole (67 mg, 0.50 mmol) in dichloromethane (10 mL). The mixture was stirred overnight at room temperature, then concentrated and the residue was diluted with ethyl acetate, washed with 2 N sodium hydroxide, water and brine, dried over magnesium sulfate and concentrated. The residue was purified by flash-chromatography (dichloromethane/methanol. 9:1) and the product was obtained as a white foam (46 mg, 20%)

### Step 2: (S)-({trans-4-[1-Amino-2-(3,3-difluoro-pyrrolidin-1-yl)-2-oxo-ethyl]-cyclohexylcarbamoyl}-methyl)-methyl-carbamic acid benzyl ester hydrochloride

The product from step 1 (40 mg, 0.071 mmol) was dissolved in ethyl acetate (3 mL), the solution was cooled to 0° C., saturated with hydrogen chloride, and stirred for 30 min at room temperature. The solvent was evaporated and the resulting white solid was dried under vacuum (20 mg, 56%). MS m/z 467 (MH⁺).

### Example 31

The hydrochloride salt of 2-{trans-4-[1-(1S)-amino-2-(3,3-difluoro-pyrrolidin-1-yl)-2-oxo-ethyl]-cyclohexylcarbamoyl}-(4R)-4-hydroxy-pyrrolidine-1-carboxylic acid benzyl ester, shown below, was prepared was prepared by the method of Example 1 using (S)-[1-(trans-4-amino-cyclohexyl)-2-(3,3-difluoro-pyrrolidin-1-yl)-2-oxo-ethyl]-carbamic acid tert-butyl ester and N-carbobenzyloxy-L-hydroxyproline. MS m/z 509 (M⁺+1).

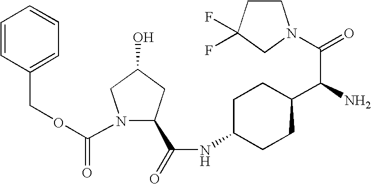

### Example 32

The hydrochloride salt of (S)-3-amino-pyrazine-2-carboxylic acid {trans-4-[1-amino-2-(3,3-difluoro-pyrrolidin-1-yl)-2-oxo-ethyl]-cyclohexyl}-amide, shown below, was prepared by the method of Example 1 using (S)-[1-(trans-4-amino-cyclohexyl)-2-(3,3-difluoro-pyrrolidin-1-yl)-2-oxo-ethyl]-carbamic acid tert-butyl ester and 3-amino-2-pyrazinecarboxylic acid. MS m/z 382 (MH⁺).

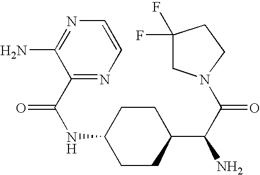

### Example 33

The hydrochloride salt of ({trans-4-[(1S)-1-amino-2-(3,3-difluoro-pyrrolidin-1-yl)-2-oxo-ethyl]-cyclohexylcarbamoyl}-methyl)-methyl-carbamic acid phenyl ester, shown below, was prepared as follows.

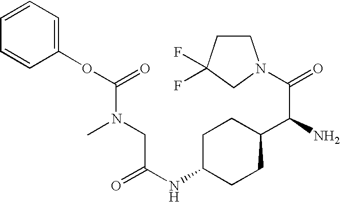

[(S)-1-{trans-4-[2-(Benzyloxycarbonyl-methyl-amino)-acetylamino]-cyclohexyl}-2-(3,3-difluoro-pyrrolidin-1-yl)-2-oxo-ethyl]-carbamic acid tert-butyl ester, Example 30, step 1, (180 mg, 0.32 mmol) was dissolved in ethanol, 40 mg of 10% palladium on carbon was added and the mixture was hydrogenated at 40 psi overnight. The mixture was filtered over diatomaceous earth and the filtrate was concentrated to dryness (133 mg, 97%).

The above product (130 mg, 0.30 mmol) and triethylamine (60 μL, 0.45 mmol) were dissolved in dichloromethane (1 mL), cooled to 0° C. and treated with a solution of phenyl chloroformate (40 μL, 0.30 mmol) in dichloromethane (2 mL). The mixture was stirred at room temperature for 2 hours, diluted with ethyl acetate, washed with 2 N hydrochloric acid, water and brine, dried over magnesium sulfate and concentrated to dryness (148 mg, 88%). The product (140 mg, 0.25 mmol) was dissolved in ether (3 mL), the solution was cooled to 0° C. and saturated with hydrogen chloride. The reaction mixture was stirred for 30 min then concentrated to dryness, leaving a white powder (75 mg, 66%) MS m/z 453 (MH⁺).

The compounds of Examples 34-51 were prepared using (S)-(trans-4-amino-cyclohexyl)-tert-butoxycarbonylamino-acetic acid methyl ester, shown below, which was synthesized as follows.

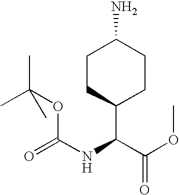

### Step 1: (S)-tert-Butoxycarbonylamino-(cis-4-hydroxy-cyclohexyl)-acetic acid methyl ester

To a solution of (S)-tert-butoxycarbonylamino-(cis-4-hydroxy-cyclohexyl)-acetic acid (10 g, 37 mmol) in DMF (80 mL) were added potassium carbonate (10.1 g, 73 mmol) and iodomethane (4.56 mL, 73 mmol), the latter drop wise. After 4 hours the mixture was poured into water and extracted with ether (2 X). The combined extracts were washed with water (3×) and brine, dried over magnesium sulfate and concentrated. The product was purified by flash-chromatography (3% methanol in dichloromethane) and isolated as a foam (5.77 g, 55%).

### Step 2. (S)-tert-Butoxycarbonylamino-(cis-4-methanesulfonyloxy-cyclohexyl)-acetic acid methyl ester

To a solution of (S)-tert-butoxycarbonylamino-(cis-4-hydroxy-cyclohexyl)-acetic acid methyl ester (5.77 g, 20 mmol) and diisopropylethylamine (7.0 mL, 40 mmol) in dichloromethane (50 mL) was added drop wise at 0° C. methanesulfonyl chloride (2.33 mL, 30 mmol). The mixture was stirred at 0° C. for 1 hour then diluted with ethyl acetate, washed with saturated sodium bicarbonate (2×), water and brine, dried over magnesium sulfate and concentrated. The product was purified by flash-chromatography (hexane/ethyl acetate, 70:30) and obtained as a foam (5.13 g, 70%).

### Step 3. (S)-(trans-4-Azido-cyclohexyl)-tert-butoxycarbonylamino-acetic acid methyl ester

To a solution of (S)-tert-butoxycarbonylamino-(cis-4-methanesulfonyloxy-cyclohexyl)-acetic acid methyl ester (5.13 g, 14 mmol) in DMF (60 mL) was added lithium azide (2.06 g, 42 mmol). The mixture was heated to 65° C. overnight, cooled, diluted with ethyl acetate, washed with water, 4% magnesium sulfate solution and brine, dried over magnesium sulfate and concentrated to dryness, leaving a colorless oil (4.07 g, 93%).

### Step 4. (S)-(trans-4-Amino-cyclohexyl)-tert-butoxycarbonylamino-acetic acid methyl ester

(S)-(trans-4-Azido-cyclohexyl)-tert-butoxycarbonylamino-acetic acid methyl ester was hydrogenated as in Step 4 of the method for preparing [(S)-1-(cis-4-amino-cyclohexyl)-2-oxo-2-pyrrolidin-1-yl-ethyl]-carbamic acid tert-butyl ester.

### Example 34

The hydrochloride salt of (S)-{[trans-4-(1-amino-2-oxo-2-pyrrolidin-1-yl-ethyl)-cyclohexylcarbamoyl]-methyl}-methyl-carbamic acid benzyl ester, shown below, was prepared as follows.

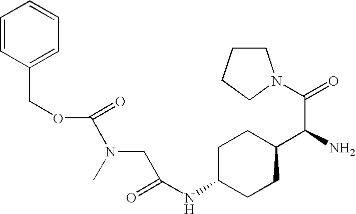

### Step 1: (S)-{trans-4-[2-(Benzyloxycarbonyl-methyl-amino)-acetylamino]-cyclohexyl}-tert-butoxycarbonylamino-acetic acid methyl ester

Prepared by coupling (S)-(trans-4-amino-cyclohexyl)-tert-butoxycarbonylamino-acetic acid methyl ester and benzyloxycarbonyl-sarcosine by the method of Example 1, step 1.

### Step 2: (S)-{trans-4-[2-(Benzyloxycarbonyl-methyl-amino)-acetylamino]-cyclohexyl}-tert-butoxycarbonylamino-acetic acid

To a solution of (S)-{trans-4-[2-(benzyloxycarbonyl-methyl-amino)-acetylamino]-cyclohexyl}-tert-butoxycarbonylamino-acetic acid methyl ester (259 mg, 0.53 mmol) in methanol (8 mL) and water (2 mL) was added 1 N sodium hydroxide (2.1 mL, 2.1 mmol). The mixture was stirred for 2 hours, concentrated to remove the methanol, diluted with water and acidified with 1 N hydrochloric acid at 0° C. The precipitate was filtered and dried (202 mg, 80%)

### Steps 3-4: (S)-([trans-4-(1-Amino-2-oxo-2-Pyrrolidin-1-yl-ethyl)-cyclohexylcarbamoyl]-methyl 1-methyl-carbamic acid benzyl ester hydrochloride

(S)-{trans-4-[2-(Benzyloxycarbonyl-methyl-amino)-acetylamino]-cyclohexyl}-tert-butoxycarbonylamino-acetic acid and pyrrolidine were coupled and the tert-butoxycarbonyl group was cleaved according to the procedures of Example 1. MS m/z 431 (MH⁺).

### Example 35

The hydrochloride salt of (S)-3-amino-pyrazine-2-carboxylic acid [trans-4-(1-amino-2-oxo-2-thiazolidin-3-yl-ethyl)-cyclohexyl]-amide, shown below, was prepared as follows. 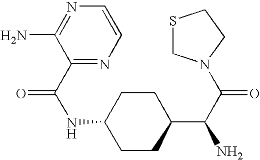
Method A

### Step 1: (S)-{trans-4-[(3-Amino-pyrazine-2-carbonyl)-amino]-cyclohexyl}-tert-butoxycarbonylamino-acetic acid methyl ester

1-(3-Dimethylaminopropyl)-3-ethylcarbodiimide hydrochloride (20.86 g, 108 mmol) was added at 0° C. to a solution of (S)-(trans-4-amino-cyclohexyl)-tert-butoxycarbonylamino-acetic acid methyl ester (26 g, 91 mmol), 3-amino-2-pyrazinecarboxylic acid (15.2 g, 109 mmol) and hydroxybenzotriazole (1407 g, 109 mmol) in dichloromethane (450 mL). The mixture was stirred overnight at room temperature, then diluted with ethyl acetate, washed with 2 N sodium hydroxide, water and brine, dried over magnesium sulfate and concentrated. The residue was purified by flash-chromatography (ethyl acetate) and the product was obtained as a foam (25.1 g, 68%).

### Step 2: (S)-{trans-4-[(3-Amino-pyrazine-2-carbonyl)-amino]-cyclohexyl}-tert-butoxycarbonylamino-acetic acid

To a solution of (S)-{trans-4-[(3-amino-pyrazine-2-carbonyl)-amino]-cyclohexyl}-tert-butoxycarbonylamino-acetic acid methyl ester (25.1 g, 62 mmol) in methanol (725 mL) and water (175 mL) was added 1 N sodium hydroxide (250 mL, 250 mmol). The mixture was stirred for 3 hours, concentrated to remove the methanol, diluted with water (350 mL) and acidified with 1 N hydrochloric acid (250 mL) at 0° C. The precipitate was collected and dried (19.7 g, 81%).

### Step 3: (S)-(1-{trans-4-[(3-Amino-pyrazine-2-carbonyl)-amino]-cyclohexyl}-2-oxo-2-thiazol idin-3-yl-ethyl)-carbamic acid tert-butyl ester

1-(3-Dimethylaminopropyl)-3-ethylcarbodiimide hydrochloride (11.5 g, 60 mmol) was added to a solution of (S)-{trans-4-[(3-amino-pyrazine-2-carbonyl)-amino]-cyclohexyl}-tert-butoxycarbonylamino-acetic acid (19.7 g, 50 mmol), thiazolidine (4.7 mL, 60 mmol) and hydroxybenzotriazole (8.1 g, 60 mmol) in dichloromethane (510 mL). The mixture was stirred 4 hours at room temperature, then was diluted with ethyl acetate (1 L), washed with 2 N sodium hydroxide, water and brine, dried over magnesium sulfate and concentrated. The residue was purified by flash-chromatography (ethyl acetate) and the product was obtained as a white foam (19 g, 82%).

### Step 4: (S)-3-Amino-pyrazine-2-carboxylic acid [trans-4-(1-Amino-2-oxo-2-thiazolidin-3-yl-ethyl)-cyclohexyl]-amide hydrochloride

(S)-(1-{trans-4-[(3-Amino-pyrazine-2-carbonyl)-amino]-cyclohexyl}-2-oxo-2-thiazolidin-3-yl-ethyl)-carbamic acid tert-butyl ester (19 g, 40.9 mmol) was dissolved in a mixture of ether (115 mL) and methanol (115 mL), the solution was cooled to 0° C., saturated with hydrogen chloride, and stirred for 10 min at room temperature. The solvent was evaporated and the resulting off-white solid was dried under vacuum (16.4 g, 100%). MS m/z 365 (MH⁺).

Method B

### Step 1: (S)-[1-(4-Hydroxy-cyclohexyl)-2-oxo-2-thiazolidin-3-yl-ethyl]-carbamic Acid tert-Butyl Ester

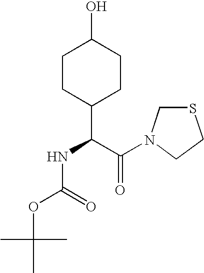

To a suspension of 30.5 g (112 mmol) of a mixture of cis and trans Boc-L-4-hydroxycyclohexylglycine (Banfi et al. Syn. Commun. 1990, 20, 3585) and 22.0 g (123 mmol) of 2-chloro-4,6-dimethoxy-1,3,5-triazine in 380 mL of isopropyl acetate at 0° C. was added 14.8 mL (134 mmol) of 4-methylmorpholine over 1 minute. This suspension was stirred at 0° C. for 2 hours and then a solution of 10.5 g (112 mmol) of thiazolidine in 9 mL of isopropyl acetate was added over 3 minutes. The resulting suspension was warmed to room temperature and held for 18 hours. The suspension was filtered and washed two times with 200 mL of 1 N Sodium Hydroxide and 200 mL of 1 N citric acid. The organic phase was washed with brine, dried over magnesium sulfate, and concentrated in vacuo to give 35.88 g of product (93%). ¹H NMR (400 MHz, CDCl₃, mixture of rotamers) 5.19-5.10 (m, 1H), 4.78-4.70 (m, 0.5H), 4.64-4.62 (m, 0.5H), 4.52-4.50 (m, 1H), 4.38-4.22 (m, 1H), 4.08-4.01 (0.5H), 3.90-3.82 (0.5H), 3.72-3.68 (m, 1H), 3.57-3.48 (m, 0.5H), 3.15-3.04 (m, 1H), 2.99-2.97 (m, 1H), 2.02-1.98 (m, 1H), 1.79-1.38 (m, 14.5H), 1.26-1.09 (m, 2H). LCMS MH⁺=345.4.

### Step 2: (S)-[2-Oxo-1-(4-oxo-cyclohexyl)-2-thiazolidin-3-yl-ethyl]-carbamic Acid tert-Butyl Ester

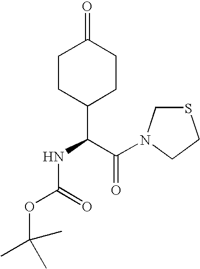

To a suspension of 36.2 g (223 mmol) sulfur trioxide pyridine complex and 200 mL (2.82 mol) dimethyl sulfoxide in 250 mL 1,2-dichloroethane at 0° C. was added a solution of 25.6 g (74.3 mmol) of (S)-[1-(4-hydroxy-cyclohexyl)-2-oxo-2-thiazolidin-3-yl-ethyl]-carbamic acid tert-butyl ester and 51.8 mL (372 mmol) of triethylamine in 500 mL of 1,2-dichloroethane. The reaction was warmed to ambient temperature and stirred for 3 hours. 1,2-Dichloroethane was removed by concentration in vacuo and replaced with 450 mL of ethyl acetate. The organic layer was washed with 225 mL of water, washed with 225 mL of 1 N hydrochloric acid, washed with brine and dried over magnesium sulfate. Concentration in vacuo gave 22.9 g (90%) of the ketone. ¹H NMR (400 MHz, CDCl₃) δ 5.22 (d, 1H, J=9 Hz), 4.71 (d, 0.5H, J=9 Hz), 4.62 (d, 0.5H, J=11 Hz), 4.49 (d, 1H, J=11 Hz), 4.38-4.35 (m, 1H), 4.05-4.01 (m, 1H), 3.99-3.87 (m, 0.5H), 3.77-3.68 (m, 1.5H), 3.10-2.98 (m, 1H), 2.42-2.23 (m, 4H), 2.11-1.96 (m, 2H), 1.56-1.34 (m, 11H).

The ketone was converted to its bisulfite adduct by the following procedure. To a solution of 25.05 g (73 mmol) ketone in 200 mL acetonitrile was added a solution of 7.61 g (73 mmol) sodium bisulfite in 40 mL of water. The resulting solution was stirred for 1 hour and then concentrated in vacuo. Acetonitrile (200 mL) was added and the resulting suspension was concentrated again to azeotrope off the water. The precipitated bisulfite adduct was then collected (25.19 g, 77%)

### Step 3: (S)-[1-(trans-4-Amino-cyclohexyl)-2-oxo-2-thiazolidin-3-yl-ethyl]-carbamic Acid tert-Butyl Ester

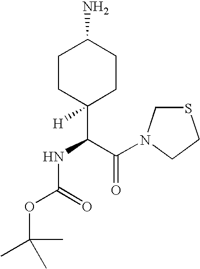

25.19 g (73 mmol) of the bisulfite adduct above was added to a solution of 17.14 g (124 mmol) potassium carbonate in 200 mL of water. This solution was extracted two times with 200 mL portions of tert-butyl methyl ether, washed with brine, dried over magnesium sulfate and concentrated in vacuo to give 22.4 g (90%) of the ketone.

6.00 g (17.5 mmol) of the ketone was mixed with 119 mL of a 4.4M ammonia solution in ethanol and stirred at ambient temperature for 1 hour. The resulting solution was added to a −50° C. suspension of 0.73 g (19.2 mmol) of sodium borohydride in 50 mL of tetrahydrofuran. After warming to 25° C. the reaction was quenched by adding 25 mL of water. The reaction was concentrated in vacuo to remove ethanol and then 100 mL of 5M sodium hydroxide and 10 g of sodium chloride is added. The resulting suspension was extracted with two 300 mL portions of tert-butyl methyl ether. The combined organics were washed with brine, dried over magnesium sulfate and concentrated in vacuo to give 5.89 g (98%) of the amine as a 5:1 (trans:cis) mixture.

The amine was dissolved in 15 mL of ethanol and added to a solution of 2.61 g (17.1 mmol) of D,L-mandelic acid in 15 mL of ethanol. The resulting solution was heated to 110° C. and 15 mL of ethanol were distilled off at atmospheric pressure. The solution was then cooled to 10° C. The mandelate salt crystallized out of solution and was collected and washed with cold ethanol. This afforded 3.87 g of a 40:1 (trans:cis) mixture of the mandelate salt of the amine.

The free amine was liberated by adding 3.75 g (7.5 mmol) of the salt to 30 mL of 5N sodium hydroxide and extracting one time with 100 mL of isopropyl acetate. After drying over magnesium sulfate, concentration in vacuo afforded 2.58 g of free amine.

### Step 4: (S)-3-Amino-pyrazine-2-carboxylic Acid [trans-4-(1-Amino-2-oxo-2-thiazol idin-3-yl-ethyl)-cyclohexyl]-amide Benzoate

0.92 mL (8.3 mmol) of N-methyl morpholine was added to a 0° C. suspension of 1.05 g (7.6 mmol) 3-aminopyrazine 2-carboxylic acid and 1.33 g (7.6 mmol) of 2-chloro-4,6-dimethoxy-1,3,5-triazine in 85 mL of isopropyl acetate. After 3 hours a solution of 2.60 g (7.6 mmol) of (S)-[1-(trans-4-amino-cyclohexyl)-2-oxo-2-thiazolidin-3-yl-ethyl]-carbamic acid tert-butyl ester in 15 mL of isopropyl acetate was added. The resulting mixture was warmed to ambient temperature and stirred for 18 hours. The reaction mixture was washed two times with 100 mL portions of 1N sodium hydroxide, washed with brine, dried over magnesium sulfate and concentrated in vacuo to give a foam. Crystallization from acetonitrile afforded 2.88 g of solid which was 98:1 (trans:cis).

Deprotection was effected by treatment with 78 mL of a 12:1 mixture of methylene chloride and trifluoroacetic acid at ambient temperature for 4 hours, followed by azeotropic distillation with methylene chloride. The residue was dissolved in methylene chloride and washed with 1N sodium hydroxide. Drying over magnesium sulfate and concentrating in vacuo afforded 2.0 g of (S)-3-amino-pyrazine-2-carboxylic acid [trans-4-(1-amino-2-oxo-2-thiazolidin-3-yl-ethyl)-cyclohexyl]-amide.

669 mg (5.5 mmol) of benzoic acid was dissolved in 2 mL of isopropanol and added to a 60° C. solution of 2.00 g (5.5 mmol) of the free base in 4 mL of isopropanol. After cooling to ambient temperature the salt crystallized from solution and was collected and dried at 70° C. to afford 1.58 g of the title compound. Mp 133.4-134.7° C. ¹H NMR (400 MHz, DMSO-d₆) 61.03-1.37 (m, 4H), 1.50-1.53 (m, 1H), 1.72-1.86 (m, 3H), 2.95 (m, 1H), 3.04 (m, 1H), 3.34 (d, J=7 Hz, 0.6H), 3.38 (d, J=7 Hz, 0.4H), 3.56-3.68 (m, 2.6H), 3.85 (m, 0.4H), 4.37 (d, J=10 Hz, 0.6H), 4.49 (d, J=10 Hz, 1H), 4.67 (d, J=9 Hz, 0.4H), 7.40-7.44 (m, 2H), 7.52 (tt, 1H), 7.76 (d, J=2 Hz, 1H), 7.89 (dd, J=8, 1 Hz, 2H), 8.14 (d, J=2 Hz, 1H), 8.36 (d, J=9 Hz, 1H). MS m/z 365 (MH+).

### Example 36

The hydrochloride salt of 3-amino-pyrazine-2-carboxylic acid {trans-4-[(1S)-1-amino-2-((2S)-2-cyano-pyrrolidin-1-yl)-2-oxo-ethyl]-cyclohexyl}-amide, shown below, was prepared by Method A of Example 35 using 3-amino-2-pyrazinecarboxylic acid and (S)-2-cyanopyrrolidine hydrochloride. MS m/z 372 (MH⁺).

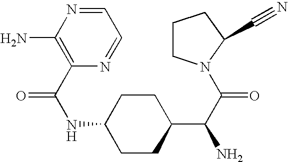

### Example 37

The hydrochloride salt of ({trans-4-[(1S)-1-amino-2-((2S)-2-cyano-pyrrolidin-1-yl)-2-oxo-ethyl]-cyclohexylcarbamoyl}-methyl)-methyl-carbamic acid benzyl ester, shown below, was prepared Method A of Example 35 using benzyloxycarbonyl-sarcosine and (S)-2-cyano pyrrolidine hydrochloride. MS m/z 456 (MH⁺).

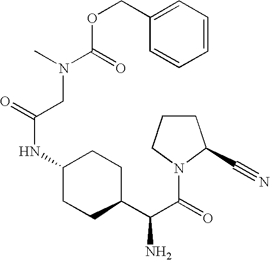

### Example 38

The hydrochloride salt of (S)-2-amino-N-[trans-4-(1-amino-2-oxo-2-pyrrolidin-1-yl-ethyl)-cyclohexyl]-nicotinamide, shown below, was prepared by Method A of Example 35 using 2-aminonicotinic acid and pyrrolidine. MS m/z 346 (MH⁺).

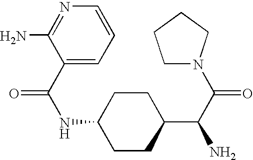

### Example 39

The hydrochloride salt of (S)-2-amino-N-[trans-4-(1-amino-2-oxo-2-thiazolidin-3-yl-ethyl)-cyclohexyl]-nicotinamide, shown below, was prepared by Method A of Example 35 using 2-aminonicotinic acid and thiazolidine. MS m/z 364 (MH⁺).

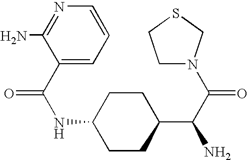

### Example 40

The hydrochloride salt of (S)-2,5-dimethyl-4-nitro-2H-pyrazole-3-carboxylic acid [trans-4-(1-amino-2-oxo-2-pyrrolidin-1-yl-ethyl)-cyclohexyl]-amide, shown below, was prepared by Method A of Example 35 using 2,5-dimethyl-4-nitro-2H-pyrazole-3-carboxylic acid and pyrrolidine. MS m/z 393 (MH⁺).

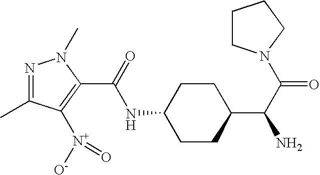

### Example 41

The hydrochloride salt of 5-[trans-4-((1S)-1-amino-2-oxo-2-pyrrolidin-1-yl-ethyl)-cyclohexyl]-5,6-dihydro-thieno[2,3-c]pyrrol-4-one, shown below, was prepared as follows. 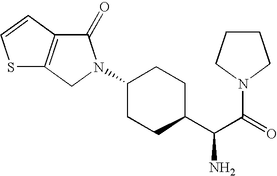
(S)-(trans-4-Amino-cyclohexyl)-tert-butoxycarbonylamino-acetic acid methyl ester, (297 mg, 1.0 mmol), and 2,3-thiophenedicarboxaldehyde (140 mg, 1.0 mmol) were mixed with xylenes (5 mL) and heated to 140° C. for 16 hours. The solution was concentrated to dryness and the product, (S)-tert-butoxycarbonylamino-[trans-4-(4-oxo-4,6-dihydro-thieno[2,3-c]pyrrol-5-yl)-cyclohexyl]-acetic acid methyl ester, was isolated by flash-chromatography (2:1 then 1:1 hexanes/ethyl acetate) as a solid (93 mg, 23%). The ester was hydrolyzed as in Example 35, Method A, Step 2. The product was coupled with pyrrolidine according to the Procedure of Example 1. Removal of the tert-butoxycarbonyl protecting group as in Example 25 gave the product as a solid. MS m/z 348 (MH⁺).

### Example 42

The hydrochloride salt of 6-[trans-4-((1S)-1-amino-2-oxo-2-pyrrolidin-1-yl-ethyl)-cyclohexyl]-6,7-dihydro-pyrrolo[3,4-b]pyridin-5-one, shown below, was prepared as follows.

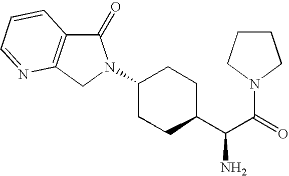

### Step 1: Ethyl 2-(Bromomethyl)nicotinate

A mixture of ethyl 2-methylnicotinate (3.1 mL, 20 mmol), azoisobutyronitrile (300 mg, 1.83 mmol) and N-bromosuccinimide (4.6 g, 26 mmol) in carbon tetrachloride (70 mL) was heated to 90° C. for 16 hours. The solution was filtered, the precipitate was washed with carbon tetrachloride, and the combined solutions were washed with saturated bicarbonate (2×) and brine, dried over magnesium sulfate and concentrated to dryness. The product was isolated by flash-chromatography (dichloromethane) as an oil (2.37 g, 49%).

### Step 2: (S)-tert-Butoxycarbonylamino-[trans-4-(5-oxo-5,7-dihydro-pyrrolo[3,4-b]pyridin-6-yl)-cyclohexyl]-acetic acid methyl ester

A solution of ethyl 2-(bromomethyl)nicotinate (976 mg, 4.0 mmol) in THF (35 mL) was added over 10 minutes to a suspension of (S)-(trans-4-amino-cyclohexyl)-tert-butoxycarbonylamino-acetic acid methyl ester and potassium carbonate (1.1 g, 8.0 mol) in THF (55 mL). After stirring for 10 min at room temperature, the mixture was concentrated to near-dryness, the residue was partitioned between ethyl acetate and water, the aqueous phase was extracted with ethyl acetate and the combined organic phases were washed with water and brine, dried over magnesium sulfate and concentrated to dryness. The product was isolated by flash-chromatography (0.5% then 1% methanol in dichloromethane) as an oil (626 mg, 39%).

### Steps 3-5: 6-[trans-4-((S)-1-Amino-2-oxo-2-pyrrolidin-1-yl-ethyl)-cyclohexyl]-6,7-dihydro-pyrrolo[3,4-b]pyridin-5-one hydrochloride

The ester was hydrolyzed as in Example 34, Step 2. The product was coupled with pyrrolidine according to the Procedure of Example 1. Removal of the Boc protecting group as in Example 25 gave the product as a solid. MS m/z 343 (MH⁺).

### Example 43

The hydrochloride salt of 6-[trans-4-((1S)-1-amino-2-oxo-2-thiazolidin-3-yl-ethyl)-cyclohexyl]-6,7-dihydro-pyrrolo[3,4-b]pyridin-5-one, shown below, was prepared as follows. 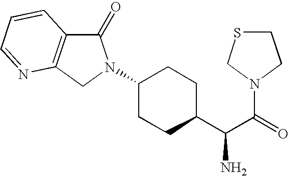
Prepared by the same sequence as Example 42, using thiazolidine. MS m/z 361 (MH⁺).

### Example 44

The hydrochloride salt of 1-{(1S)-amino-[trans-4-(5-oxo-5,7-dihydro-pyrrolo[3,4-b]pyridin-6-yl)-cyclohexyl]-acetyl)-(2S)-pyrrolidine-2-carbonitrile, shown below, was prepared as follows. 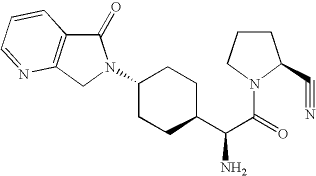
Prepared by the same sequence as Example 42 using 2-cyanopyrrolidine hydrochloride. MS m/z 368 (MH⁺).

### Example 45

The hydrochloride salt of 6-{trans-4-[(1S)-1-amino-2-(3,3-difluoro-pyrrolidin-1-yl)-2-oxo-ethyl]-cyclohexyl}-6,7-dihydro-pyrrolo[3,4-b]pyridin-5-one, shown below, was prepared using the method of Example 42, using 3,3-difluoropyrrolidine hydrochloride. MS m/z 379 (MH⁺).

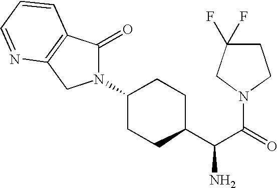

### Example 46

The hydrochloride salt of 6-[trans-4-((1S)-1-amino-2-oxo-2-pyrrolidin-1-yl-ethyl)-cyclohexyl]-6,7-dihydro-pyrrolo[3,4-b]pyrazin-5-one, shown below, was prepared as follows.

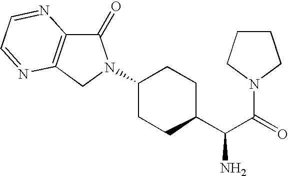

### Step 1. 3-Methylpyrazine-2-carboxylic acid ethyl ester

A solution of 3-methylpyrazinecarboxylic acid (Vishweshar, J. Org. Chem. 2002, 67, 556) (3.2 mL, 30 mmol) in ethanol (20 mL) was saturated with hydrogen chloride and stirred at 70° C. for 16 hours. The solution was concentrated, the residue was taken up in chloroform and the solution was washed with 1 N sodium hydroxide and brine, dried over magnesium sulfate and concentrated to dryness, leaving a brown solid (828 mg, 66%)

### Step 2-. 3-Bromomethyl-Pyrazine-2-carboxylic acid ethyl ester

Prepared from 3-methylpyrazine-2-carboxylic acid ethyl ester and (S)-(trans-4-amino-cyclohexyl)-tert-butoxycarbonylamino-acetic acid methyl ester by the same sequence as in Example 41, using pyrrolidine.

### Steps 3-6: 6-[trans-4-((S)-1-Amino-2-oxo-2-pyrrolidin-1-yl-ethyl)-cyclohexyl]-6,7-dihydro-pyrrolo[3,4-b]pyrazin-5-one

Performed as in Example 42. MS m/z 344 (MH⁺).

### Example 47

The hydrochloride salt of pyrrolo[1,2-c]pyrimidine-3-carboxylic acid {trans-4-[(1S)-1-Amino-2-((2S)-2-cyano-pyrrolidin-1-yl)-2-oxo-ethyl]-cyclohexyl}-amide, shown below, was prepared as follows.

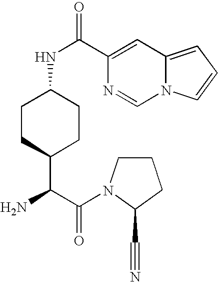

### Steps 1-4: [(S)-1-(trans-4-Amino-cyclohexyl)-2-((S)-2-carbamoyl-pyrrolidin-1-yl)-2-oxo-ethyl]-carbamic acid tert-butyl ester

(S)-tert-Butoxycarbonylamino-(cis-4-hydroxy-cyclohexyl)-acetic acid and L-prolinamide were coupled and the product transformed into the title product according to Steps 1-4 of the method for making (S)-(trans-4-Amino-cyclohexyl)-tert-butoxycarbonylamino-acetic acid methyl ester.

### Step 5: (2-((S)-2-Carbamoyl-pyrrolidin-1-yl)-2-oxo-(S)-1-{trans-4-[(pyrrolo[1,2-c]pyrimidine-3-carbonyl)-amino]-cyclohexyl}-ethyl)-carbamic acid tert-butyl ester

The product of step 4 was coupled with pyrrolo[1,2-c]pyrimidine-3-carboxylic acid (Minguez et al, Tetrahedron Lett. 1996, 37, 4263) according to the procedure of Example 1, step 1.

### Step 6: (2-((S)-2-Cyano-pyrrolidin-1-yl)-2-oxo-(S)-1-{trans-4-[(pyrrolo[1,2-c]pyrimidine-3-carbonyl)-amino]-cyclohexyl}-ethyl)-carbamic acid tert-butyl ester

The product of step 5 (128 mg, 0.25 mmol) and imidazole (34 mg, 0.5 mmol) were dissolved in dichloromethane (5 mL) and pyridine (0.5 mL). The solution was cooled to 0° C. and a solution of phosphorus oxychloride (93 μL, 1.0 mmol) in dichloromethane (2 mL) was added over 10 minutes. After 1.5 hours at 0° C. additional dichloromethane (2 mL), pyridine (1 mL) and phosphorus oxychloride (30 μL, 0.3 mmol) were added and the solution was stirred overnight, then concentrated to dryness. The residue was triturated with ethyl acetate and filtered. The filtrate was concentrated and the residue was purified by preparative thin-layer chromatography (dichloromethane/methanol, 9:1). Yield: 20 mg (16%).

### Step 7: (Pyrrolo[1,2-c]pyrimidine-3-carboxylic acid {trans-4-[(1S)-1-Amino-2-((2S)-2-cyano-pyrrolidin-1-yl)-2-oxo-ethyl]-cyclohexyl}-amide hydrochloride

The product of step 6 was treated with HCl in dioxane as in Example 25. MS m/z 395 (MH⁺).

### Example 48

The hydrochloride salt of (S)-4-amino-2,5-dimethyl-2H-pyrazole-3-carboxylic acid [trans-4-(1-amino-2-oxo-2-pyrrolidin-1-yl-ethyl)-cyclohexyl]-amide, shown below, was prepared as follows. 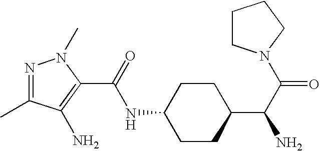
(S)-2,5-Dimethyl-4-nitro-2H-pyrazole-3-carboxylic acid [trans-4-(1-amino-2-oxo-2-pyrrolidin-1-yl-ethyl)-cyclohexyl]-amide, Example 40 (85 mg, 0.20 mmol) was dissolved in methanol (3 mL) containing 10% palladium on carbon (20 mg) and hydrogenated at 45 psi for 16 hours. The solution was filtered over diatomaceous earth and concentrated to a solid (10 mg, 88%). MS m/z 363 (MH⁺).

### Example 49

The hydrochloride salt of 3-amino-pyrazine-2-carboxylic acid {trans-4-[(1S)-1-amino-2-oxo-2-(1-oxo-1×4-thiazolidin-3-yl)-ethyl]-cyclohexyl}-amide, shown below, was prepared as follows.

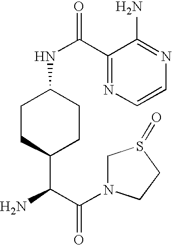

A solution of (S)-3-amino-pyrazine-2-carboxylic acid [trans-4-(1-amino-2-oxo-2-thiazolidin-3-yl-ethyl)-cyclohexyl]-amide, Example 35 (200 mg, 0.5 mmol), in trifluoroacetic acid (1.5 mL) was cooled to 0° C. and treated with peroxytrifluoroacetic acid (0.13 mL of a 4.0 M solution, 0.5 mmol). The mixture was stirred at 0° C. for 3 hours then diluted with ether. The precipitate was collected, washed with ether and dried (130 mg, 68%). MS m/z 381 (MH⁺).

### Example 50

The hydrochloride salt of (2S)-2-amino-2-[trans-4-(1,1-dioxo-1,3-dihydro-1λ⁶-benzo[d]isothiazol-2-yl)-cyclohexyl]-1-pyrrolidin-1-yl-ethanone, shown below, was prepared as follows.

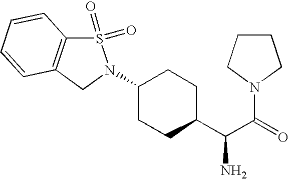

### Step 1: 2-Bromomethyl-benzenesulfonyl Chloride

Azoisobutyronitrile (60 mg) was added to a solution of 2-methylphenylsulfonyl chloride (4.3 mL, 30 mmol) and N-bromosuccinimide (5.3 g, 30 mmol) in carbon tetrachloride (50 mL). The mixture was heated at 90° C. for 16 hours then concentrated to dryness. The residue was distilled under high vacuum, to give the product (bp 78-84° C. at 0.1 mm Hg) as an oil (837 mg, 10%).

### Step 2: (S)-Amino-[trans-4-(1,1-dioxo-1,3-dihydro-1λ⁶-benzo[d]isothiazol-2-yl)-cyclohexyl]-acetic acid methyl ester

To a suspension of (S)-(trans-4-amino-cyclohexyl)-tert-butoxycarbonylamino-acetic acid methyl ester, (890 mg, 3 mmol), and potassium carbonate (871 mg, 6.3 mmol) in DMF (5 mL) was added 2-bromomethyl-benzenesulfonyl chloride (808 mg, 3.0 mmol) over 2 hours. After 16 hours at room temperature the mixture was diluted with ethyl acetate, washed with water, 4% aqueous magnesium sulfate (3×) and brine, dried over magnesium sulfate and concentrated. The product was isolated by flash-chromatography (1% to 4% gradient of methanol in dichloromethane) followed by a second flash-chromatography (hexanes/ethyl acetate, 3:1 then 2:1) as a solid (112 mg, 8.5%).

### Steps 3-5. (2S)-2-Amino-2-[trans-4-(1,1-dioxo-1,3-dihydro-1λ⁶-benzo[d]isothiazol-2-yl)-cyclohexyl]-1-pyrrolidin-1-yl-ethanone hydrochloride

The ester was hydrolyzed as in Example 34, Step 2. The product was coupled with pyrrolidine according to the Procedure of Example 1. Removal of the tert-butoxycarbonyl protecting group as in Example 25 gave the product as a solid. MS m/z 478 (MH⁺).

### Example 51

The hydrochloride salt of (2S)-2-amino-2-[trans-4-(1,1-dioxo-1,3-dihydro-1λ⁶-benzo[d]isothiazol-2-yl)-cyclohexyl]-1-thiazolidin-3-yl-ethanone, shown below, was prepared using the method of Example 50 with the exception that thiazolidine was used in step 4. MS m/z 496 (MH⁺).

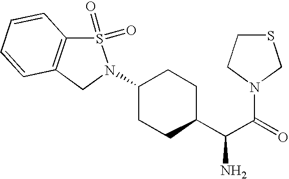

### Examples 52-53

The compounds of Examples 52-53 were prepared using [(1S)-1-(trans-4-amino-cyclohexyl)-2-((R)-3-fluoro-pyrrolidin-1-yl)-2-oxo-ethyl]-carbamic acid tert-butyl ester, shown below, was prepared from (S)-tert-butoxycarbonylamino-(cis-4-hydroxy-cyclohexyl)-acetic acid and (R)-3-fluoropyrrolidine hydrochloride (Giardina, G. et al, Synlett 1995, 55) using the method for making (S)-[1-(trans-4-amino-cyclohexyl)-2-oxo-2-pyrrolidin-1-yl-ethyl]-carbamic acid tert-butyl ester.

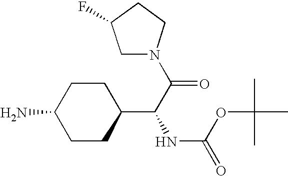

### Example 52

The hydrochloride salt of 6-hydroxy-pyridine-2-carboxylic acid {trans-4-[(1S)-1-amino-2-((3R)-3-fluoro-pyrrolidin-1-yl)-2-oxo-ethyl]-cyclohexyl}-amide, shown below, was prepared from [(S)-1-(trans-4-amino-cyclohexyl)-2-((R)-3-fluoro-pyrrolidin-1-yl)-2-oxo-ethyl]-carbamic acid tert-butyl ester and 6-hydroxypicolinic acid by the method of Example 1. MS m/z 365 (MH⁺).

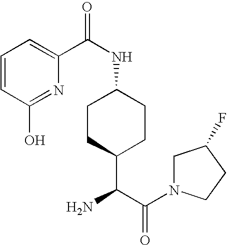

### Example 53

The hydrochloride salt of 6-Hydroxy-pyrazine-2-carboxylic acid {trans-4-[(1S)-1-Amino-2-((3R)-3-fluoro-pyrrolidin-1-yl)-2-oxo-ethyl]-cyclohexyl}-amide hydrochloride, shown below, was prepared from [(S)-1-(trans-4-amino-cyclohexyl)-2-((R)-3-fluoro-pyrrolidin-1-yl)-2-oxo-ethyl]-carbamic acid tert-butyl ester and 6-hydroxypyrazine-2-carboxylic acid by the method of Example 1. MS m/z 366 (MH⁺).

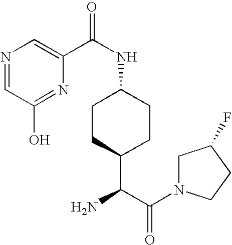

### Examples 54-62

The compounds of Examples 54-62 were prepared using tert-butyl (1S)-1-(trans-4-aminocyclohexyl)-2-[(2S)-2-cyanopyrrolidin-1-yl]-2-oxoethylcarbamate, shown below, was synthesized as follows.

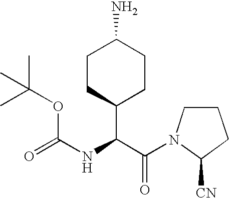

### Step 1: Methyl (2S)-(trans-4-{[(benzyloxy)carbonyl]amino}cyclohexyl)[(tert-butoxycarbonyl)amino]acetate

A solution of benzyl chloroformate (0.07 mL, 0.5 mmol) in dichloromethane was added drop wise to a mixture of (S)-(trans-4-amino-cyclohexyl)-tert-butoxycarbonylamino-acetic acid methyl ester (143 mg, 0.5 mmol), diisopropylethylamine (0.09 mL, 0.5 mmol) and 4-dimethylaminopyridine (DMAP) (5 mg) in dichloromethane at 0° C. The reaction was allowed to warm to room temperature. After 2 hours, the mixture was washed sequentially with 10% NaHCO₃ solution and brine, dried over magnesium sulfate, filtered and concentrated. The reaction was repeated using benzyl chloroformate (0.74 mL, 5.25 mmol), (S)-(trans-4-amino-cyclohexyl)-tert-butoxycarbonylamino-acetic acid methyl ester (1.43 g, 5.0 mmol), diisopropylethylamine (0.9 mL, 5.25 mmol) and DMAP (5 mg). The combined crude products were purified by chromatography (Biotage 40S (Dyax Corp., Charlottesville, Va.), 40% ethyl acetate/hexanes) to provide 1.84 g of the title compound.

### Step 2: (2S)-(trans-4-{[(Benzyloxy)carbonyl]amino}cyclohexyl)[(tert-butoxycarbonyl)amino]acetic acid

To a solution of methyl (2S)-(trans-4-{[(benzyloxy)carbonyl]amino}cyclohexyl)[(tert-butoxycarbonyl)amino]acetate (1.77 g, 4.2 mmol) in 20 mL methanol/THF (1:1), was added a solution of sodium hydroxide (8.4 mL, 1N). After 2.5 hours, the reaction was concentrated. The residue was diluted in water and the pH adjusted to pH 3 with 2N hydrochloric acid solution. The white precipitate was filtered and dried to provide 1.51 g of the title compound.

### Step 3: 1-[(2S)-2-(trans-4-{[(Benzyloxy)carbonyl]amino}cyclohexyl)-2-[(tert-butoxycarbonyl)amino]ethanoyl}-L-prolinamide

To a solution of (2S)-(trans-4-{[(benzyloxy)carbonyl]amino}cyclohexyl)[(tert-butoxycarbonyl)amino]acetic acid (1.42 g, 3.5 mmol) and L-prolinamide (0.42 g, 3.68 mmol) in 20 mL dichloromethane, was added hydroxybenzotriazole (0.52 g, 3.85 mmol) and 1-(3-dimethylaminopropyl)-3-ethylcarbodiimide hydrochloride (0.70 g, 3.68 mmol). After 8 hours, saturated NaHCO₃ solution was added and mixture was extracted with ethyl acetate. The combined organic layers were washed with brine, dried over magnesium sulfate, filtered and concentrated. The crude product was purified by chromatography (Biotage 40M (Dyax Corp., Charlottesville, Va.), 9:1 dichloromethane/methanol) to provide 1.65 g of the title compound.

### Step 4: Benzyl trans-4-((1S)-1-[(tert-Butoxycarbonyl)amino]-2-[(2S)-2-cyanopyrrolidin-1-yl]-2-oxoethyl]cyclohexylcarbamate

A solution of phosphorous oxychloride (0.93 mL, 10 mmol) in dichloromethane was added drop wise to a solution of 1-{(2S)-2-(4-{[(benzyloxy)carbonyl]amino}cyclohexyl)-2-[(tert-butoxycarbonyl)amino]ethanoyl}-L-prolinamide (1.25 g, 2.5 mmol) and imidazole (0.30 g, 5 mmol) in 30 mL dichloromethane and 3 mL pyridine at 0° C. After 3 hours at 0° C., the mixture was concentrated to dryness, triturated with ethyl acetate, filtered through diatomaceous earth, dried over magnesium sulfate and concentrated. The crude product was purified by chromatography (Biotage 40S (Dyax Corp., Charlottesville, Va.), 95:5 dichloromethane/methanol) to provide 1.07 g of the title compound.

### Step 5: tert-Butyl (1S)-1-(trans-4-Aminocyclohexyl)-2-[(2S)-2-cyanopyrrolidin-1-yl]-2-oxoethylcarbamate

To a solution of benzyl 4-{(1S)-1-[(tert-butoxycarbonyl)amino]-2-[(2S)-2-cyanopyrrolidin-1-yl]-2-oxoethyl}cyclohexylcarbamate (1.07 g, 2.2 mmol) in 30 mL absolute ethanol, was added 10% Pd/C (1.0 g) followed by 1,4-cyclohexadiene (2.1 mL, 22 mmol). After 2 hours, the mixture was filtered through diatomaceous earth and concentrated to dryness to provide 0.79 g of the title compound.

### Example 54

N-(trans-4-{(1S)-1-Amino-2-[(2S)-2-cyanopyrrolidin-1-yl]-2-oxoethyl}cyclohexyl)-1-methyl-1H-pyrrolo[2,3-c]pyridine-5-carboxamide hydrochloride, shown below, was prepared as follows.

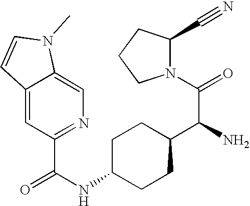

Diisopropylethylamine (0.067 mL, 0.385 mmol) was added drop wise to tert-butyl (1S)-1-(trans-4-aminocyclohexyl)-2-[(2S)-2-cyanopyrrolidin-1-yl]-2-oxoethylcarbamate, (90 mg, 0.26 mmol), 1-methyl-1H-pyrrolo[2,3-c]pyridine-5-carboxylic acid (45 mg, 0.26 mmol, prepared according to WO 02/100857) and HATU (117 mg, 0.31 mmol) in 5 mL dichloromethane at 0° C. The reaction was allowed to warm to room temperature. After 18 hours, saturated NaHCO₃ solution was added and mixture was extracted with ethyl acetate. The combined extracts were washed with brine, dried over magnesium sulfate, filtered and concentrated. The crude product was purified by chromatography (Biotage 40S (Dyax Corp., Charlottesville, Va.), 98:2 dichloromethane/methanol) to provide 115 mg of tert-butyl (1S)-2-[(2S)-2-cyanopyrrolidin-1-yl]-1-(trans-4-{[(1-methyl-1H-pyrrolo[2,3-c]pyridin-5-yl)carbonyl]amino}cyclohexyl)-2-oxoethylcarbamate as a white powder.

The solid was treated with HCl in dioxane as described in Example 25, to give 57 mg of the product as a light yellow solid. MS m/z 409 (MH⁺).

### Example 55

The hydrochloride salt of N-(trans-4-{(1S)-1-amino-2-[(2S)-2-cyanopyrrolidin-1-yl]-2-oxoethyl}cyclohexyl)-3,3-dimethyl-2,3-dihydrofuro[2,3-c]pyridine-5-carboxamide, shown below, was prepared by the method of Example 54 using tert-butyl (1S)-1-(trans-4-aminocyclohexyl)-2-[(2S)-2-cyanopyrrolidin-1-yl]-2-oxoethylcarbamate and 3,3-dimethyl-2,3-dihydrofuro[2,3-c]pyridine-5-carboxylic acid (prepared according to WO 02/100857) to give 64 mg of a solid. MS m/z 426 (MH⁺).

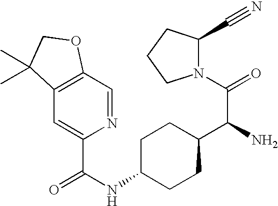

### Example 56

The hydrochloride salt of N-(trans-4-{(S)-1-amino-2-[(2S)-2-cyanopyrrolidin-1-yl]-2-oxoethyl}cyclohexyl)pyrrolo[1,2-a]pyrazine-3-carboxamide, shown below, was prepared by the method of Example 54 using tert-butyl (1S)-1-(trans-4-aminocyclohexyl)-2-[(2S)-2-cyanopyrrolidin-1-yl]-2-oxoethylcarbamate and pyrrolo[1,2-a]pyrazine-3-carboxylic acid (prepared according to WO 03/070732) to give 66 mg of a solid. MS m/z 395 (MH⁺). 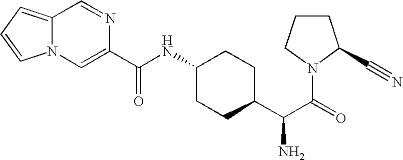
Prepared from tert-butyl (1S)-1-(trans-4-aminocyclohexyl)-2-[(2S)-2-cyanopyrrolidin-1-yl]-2-oxoethylcarbamate and pyrrolo[1,2-a]pyrazine-3-carboxylic acid (prepared according to WO 03/070732) as in Example 54, to give 66 mg of a solid. MS m/z 395 (MH⁺).

### Example 57

The hydrochloride salt of N-(trans-4-{(1S)-1-amino-2-[(2S)-2-cyanopyrrolidin-1-yl]-2-oxoethyl}cyclohexyl)thieno[3,2-c]pyridine-6-carboxamide, shown below, was prepared by the method of Example 54 using tert-butyl (1S)-1-(trans-4-aminocyclohexyl)-2-[(2S)-2-cyanopyrrolidin-1-yl]-2-oxoethylcarbamate and thieno[3,2-c]pyridine-6-carboxylic acid (prepared according to WO 02/100857) to give 82 mg of a solid. MS m/z 412 (MH⁺).

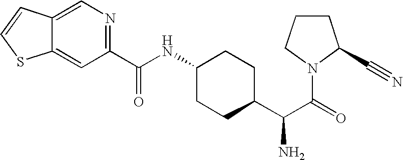

### Example 58

The hydrochloride salt of N-(trans-4-{(1S)-1-amino-2-[(2S)-2-cyanopyrrolidin-1-yl]-2-oxoethyl}cyclohexyl) furo[2,3-c]pyridine-5-carboxamide, shown below, was prepared by the method of Example 54 using tert-butyl (1S)-1-(trans-4-aminocyclohexyl)-2-[(2S)-2-cyanopyrrolidin-1-yl]-2-oxoethylcarbamate and furo[2,3-c]pyridine-5-carboxylic acid (prepared according to WO 02/100857), to give 58 mg of a solid. MS m/z 396 (MH⁺).

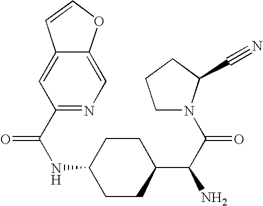

### Example 59

The hydrochloride salt of N-(trans-4-{(1S)-1-amino-2-[(2S)-2-cyanopyrrolidin-1-yl]-2-oxoethyl}cyclohexyl)-5,7-dimethylpyrrolo[1,2-c]pyrimidine-3-carboxamide, shown below, was prepared by the method of Example 54 using tert-butyl (1S)-1-(trans-4-aminocyclohexyl)-2-[(2S)-2-cyanopyrrolidin-1-yl]-2-oxoethylcarbamate and 5,7-dimethylpyrrolo[1,2-c]pyrimidine-3-carboxylic acid (prepared from 3,5-dimethylpyrrole-2-carboxaldehyde according to J. Org. Chem (1999), 64, 7788) as in Example 54, to give 47 mg of a solid. MS m/z 423 (MH⁺).

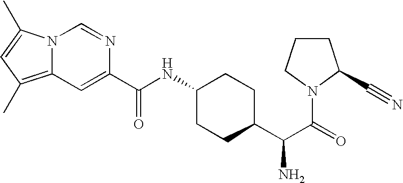

### Example 60

The hydrochloride salt of N-(trans-4-{(1S)-1-amino-2-[(2S)-2-cyanopyrrolidin-1-yl]-2-oxoethyl}cyclohexyl)thieno[2,3-c]pyridine-5-carboxamide, shown below, was prepared by the method of Example 54 using tert-butyl (1S)-1-(trans-4-aminocyclohexyl)-2-[(2S)-2-cyanopyrrolidin-1-yl]-2-oxoethylcarbamate and thieno[2,3-c]pyridine-5-carboxylic acid (prepared according to WO 02/100857) as in Example 54, to give 85 mg of a solid. MS m/z 412 (MH⁺).

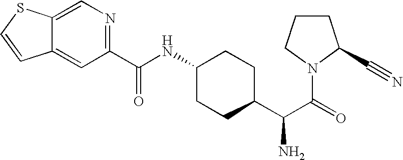

### Example 61

The hydrochloride salt of N-(trans-4-{(1S)-1-amino-2-[(2S)-2-cyanopyrrolidin-1-yl]-2-oxoethyl}cyclohexyl)furo[3,2-c]pyridine-6-carboxamide, shown below, was prepared by the method of Example 54 using tert-butyl (1S)-1-(trans-4-aminocyclohexyl)-2-[(2S)-2-cyanopyrrolidin-1-yl]-2-oxoethylcarbamate and furo[3,2-c]pyridine-6-carboxylic acid (prepared according to WO 02/100857), to give 46 mg of a solid. MS m/z 396 (MH⁺).

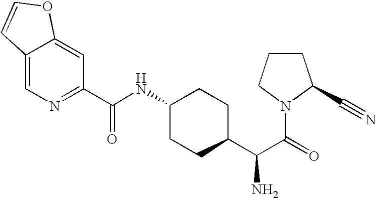

### Example 62

The hydrochloride salt of N-(trans-4-{(S)-1-amino-2-[(2S)-2-cyanopyrrolidin-1-yl]-2-oxoethyl}cyclohexyl)-3-ethynylfuro[2,3-c]pyridine-5-carboxamide, shown below, was prepared by the method of Example 54 using tert-butyl (1S)-1-(trans-4-aminocyclohexyl)-2-[(2S)-2-cyanopyrrolidin-1-yl]-2-oxoethylcarbamate and 3-ethynylfuro[3,2-c]pyridine-6-carboxylic acid (prepared according to WO 02/100857) as in Example 54, to give 48 mg of a solid. MS m/z 420 (MH⁺). 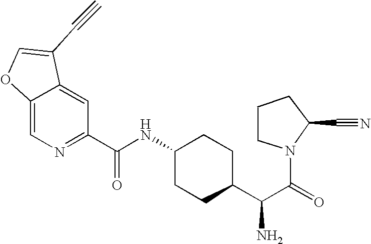
Biological Protocols

The utility of the compounds of Formula (I), the stereoisomers and prodrugs thereof, and the pharmaceutically acceptable salts of the compounds, stereoisomers, and prodrugs, in the treatment or prevention of diseases (such as are detailed herein) in animals, particularly mammals (e.g., humans) may be demonstrated by the activity thereof in conventional assays known to one of ordinary skill in the relevant art, including the in vitro and in vivo assays described below. Such assays also provide a means whereby the activities of the compounds of Formula (I) can be compared with the activities of other known compounds.

In Vitro Assay for Dipeptidyl Peptidase Inhibition

The inhibition of dipeptidyl peptidase was demonstrated in vitro for hydrochloride salts of compounds of the present invention, particularly those of Examples 1-33 and 35-62, by using the following assay, which is adapted from published methods for the measurement of DPP-IV activity (Assay of dipeptidyl peptidase IV in serum by fluorimetry of 4-methoxy-2-naphthylamide. (1988) Scharpe, S., DeMeester, I., Vanhoof, G., Hendriks, D., Van Sande, M., Van Camp, K. and Yaron, A. Clin. Chem. 34:2299-2301; Dipeptidyl peptidases of human lymphocytes (1988) Lodja, Z. Czechoslovak Medicine, 11: 181-194.). 150 μL of an enzyme-substrate solution was pipetted into microtiter wells of a polystyrene 96-well plate, and maintained at 4° C. The enzyme-substrate solution comprised 50 μM Gly-Pro-4-methoxy B naphthylamide HCl in 50 mM Tris assay buffer pH 7.3 containing 0.1M sodium chloride, 0.1% (v/v) Triton and 50 μU/mL DPP-IV (Enzyme Systems Products Cat#SPE-01, DPP-IV 5 mU/mL stock). 5 μL/well of compounds of Formula (I) were added, bringing the final compound of Formula (I) concentrations to 3 μM-10 nM per well.

Controls. Enzyme was omitted from four (4) wells, as a reagent blank. 5 μL of 3 mM Diprotin A was added to four wells as a positive quality control, providing a final Diprotin A concentration of 100 μM. To measure total enzyme activity (i.e. a negative control), without the influence of any compounds of Formula (I), 5 μL of distilled water was added to four wells.

The entire assay was incubated overnight (about 14-18 hours) at 37° C. The reaction was quenched by adding 10 μL of Fast Blue B solution (0.5 mg/mL Fast Blue B in a buffer comprising 0.1 M sodium acetate pH 4.2 and 10% (v/v) Triton X-100 to each well, followed by shaking for approximately 5 minutes at room temperature. The plates were analyzed on a Spectramax spectrophotometer, or equivalent equipment, (absorption maximum at 525 nm). IC₅₀ data for compounds were obtained by measuring the activity of DPP-IV over a range of compound concentrations. Compounds of the present invention were found to have IC₅₀ (concentration of test compound required for 50% inhibition) values between about 0.3 nM and about 1 μM or less. Compounds of Examples 1-3, 6 and 7 had IC₅₀ values of greater than 500 nM and less than or equal to 1 μM. Preferred compounds of the present invention were found to have IC₅₀ values of 500 nM or less. Compounds of Examples 4-5, 8, 14, 17, 21, 28, 34 and 49 had IC₅₀ values of greater than 100 nM and less than or equal to 500 nM. More preferred compounds of the present invention, such as the compounds of Examples 9-13, 15-16, 18-20, 22-27, 29, 30-33, 36-39, 41-48, 50-62 were found to have IC₅₀ values of 100 nM, or less. For example, the compound of Example 23 had an IC₅₀ value of 18 nM.

In Vivo Assay for Glucose Lowering

The glucose lowering effects of DPP-IV inhibitors for some compounds of the present invention were exemplified in 4-6 week old KK/H1J mice (Jackson Labs) in the context of an oral glucose tolerance test. Oral glucose tolerance tests (“OGTT”) have been in use in humans since, at least, the 1930s, Pincus et al., Am. J. Med. Sci, 188: 782 (1934), and are routinely used in the diagnosis of human diabetes, though not to evaluate the efficacy of therapeutic agents in patients. KK mice have been used to evaluate glitazones (Fujita et al. Diabetes 32:804-810 (1983); Fujiwara et al., Diabetes 37: 1549-48 (1988); Izumi et al. Biopharm Drug. Dispos. 18:247-257 (1997)), metformin (Reddi et al. Diabet. Metabl. 19:44-51 (1993)), glucosidase inhibitors (Hamada et al. Jap. Pharmacol. Ther. 17:17-28 (1988); Matsuo et al. Am. J. Clin. Nutr. 55:314S-317S (1992)), and the extra-pancreatic effects of sulfonylureas (Kameda et al Arzneim. Forsch./Drug Res. 32:39044 (1982); Muller et al. Horm. Metabl. Res. 28:469-487 (1996)).

KK mice are derived from an inbred line first established by Kondo et al. (Kondo et al. Bull. Exp. Anim. 6:107-112 (1957)). The mice spontaneously develop a hereditary form of polygenic diabetes that progresses to cause renal, retinal and neurological complications analogous to those seen in human diabetic subjects, but they do not require insulin or other medication for survival.

The mice were fasted overnight (about 14-18 hours), but allowed free access to water. After fasting, (time “t”=0), 25 μL of blood was drawn from the retro-orbital sinus and added to 0.025% heparinized saline (100 μL) on ice. The mice (10 per group) were then orally dosed with a solution of a compound of the present invention in 0.5% methylcellulose (0.2 mL/mouse). Two controls groups received only 0.5% methylcellulose. At t=15 minutes, the mice were bled, as described above, and then dosed with 1 mg/kg glucose in distilled water (0.2 mL/mouse). The first control group was dosed with glucose. The second control group was dosed with water. At t=45 minutes, the mice were again bled, as described above. The blood samples were centrifuged, the plasma collected and analyzed for glucose content on a Roche-Hitachi 912 glucose analyzer. The data is expressed as percent (%) inhibition of glucose excursion relative to the two control groups (i.e. the glucose level in the animals receiving glucose but no test compound representing 0% inhibition and the glucose concentration in the animals receiving only water representing 100% inhibition). Typically, the compounds of the present invention that were tested in this assay demonstrated glucose lowering. For example, the compound of Example 23 exhibited an inhibition of about 65% inhibition at a dose level of 5 mg/kg. However, a few compounds, such as the compound of Example 26 did not demonstrate inhibition in this assay.

Madin-Darby Canine Kidney Cells (MDCK) Permeability Assay

Drug permeability through MDCK cell monolayers is known to be a useful tool as a model for cellular barrier for assessing intestinal epithelial drug transport, and thus to estimate intestinal permeability for orally administered compounds.

In this assay, monolayer cultures, suitable for investigation of compound permeability, were grown on Falcon/BD 96-well membrane inserts. The monolayers were maintained at 37° C. in a 5% CO₂ atmosphere at 95% relative humidity until confluent. Apparent permeabilities of test compounds were determined in duplicate at a single concentration of 2 μM in the apical to basolateral direction. The transport investigations were initiated by the addition of the compound (2 μM of a solution containing 0.4% DMSO) to the apical compartment and the plates were maintained under culture conditions during the course of the experiment. The basolateral compartments following 2.5 hours of exposure and the final apical compartments were collected and analyzed for compound content by LC-MS/MS. The apparent permeability of each compound was then calculated.

Compounds of the present invention, such as those of Examples 18 and 27 were used in this assay. By comparison, comparison DPP-IV inhibitor compounds, which are generically disclosed in International Application WO02/076450, were also synthesized, and then used in this assay. The results of this assay, provided below, show that the compounds of the present invention are predicted to have substantially better gastrointestinal premeabilities that similar compounds generically disclosed within International Application WO02/076450.

| | | | Permeability |
|---|---|---|---|
| | Permeability | Compounds of the | (10⁻⁶ |
| Comparison Compounds | (10⁻⁶ cm/sec) | Present Invention | cm/sec) |
| 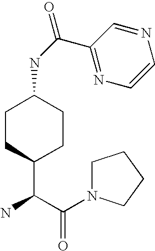 | 1.1 | Example 18 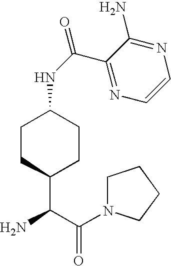 | 4.8 |
| 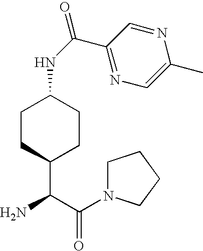 | 0.7 | Example 27 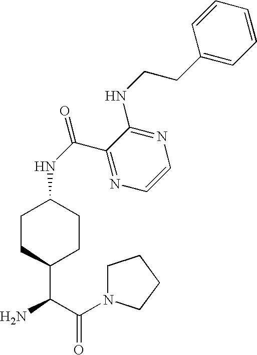 | 6.2 |

1. A compound of having the formula 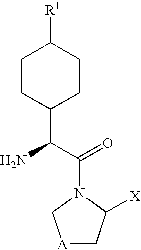
or a prodrug thereof, or a pharmaceutically acceptable salt of said compound or prodrug, or a solvate of said compound, prodrug or salt, wherein:
X is H or —CN;
A is CH₂, CHF, CF₂ or S(O)ₙ;
n is 0, 1 or 2;
R¹ is —NR²R³, Het^{(I)}, or Het^{(II)};
R² is —C(O)R⁴, —SO₂R⁴, —C(O)NHR⁴, or —COOR⁴;
R³ is H, C₁₋₆alkyl, or C₃₋₈cycloalkyl;
R⁴ is selected from the group consisting of
(a) Het^{(I)}-C₀₋₆alkylenyl-,
(b) Het^{(II)}-C₀₋₆alkylenyl-,
(c) R⁵OC(O)N(R⁶)-C₀₋₆alkylenyl-,
(d) R⁵C(O)N(R⁶)-C₁₋₆alkylenyl-,
(e) phenyl-C₀₋₆alkylenyl-amino-C₀₋₆alkylenyl-,
(f) phenylsulfonyl-C₁₋₆alkylenyl-,
(g) phenylthio-C₁₋₆alkylenyl-,
(h) naphthyloxy-C₁₋₆alkylenyl-, and
(i) C₃₋₈cycloalkyl- wherein said C₃₋₈cycloalkyl is optionally substituted with C₁₋₆alkyl, C₁₋₆alkoxy, hydroxy, halo, or phenyl optionally substituted with one to three halo; OKHet^{(I)} is oxazolidinyl, 2,3-dihydro-1H-pyrrolo[3,4-b]pyridyl, 6,7-dihydro-5H-pyrrolo[3,4-b]pyrazinyl, 6,7-dihydro-5H-pyrrolo[3,4-b]pyridyl, 2,3-dihydro-1H-pyrrolo[3,4-c]pyridyl, 5,6-dihydro-4H-thieno [2,3-c]pyrrolyl, pyrrolo[1,2-c]pyrimidyl, 1H-pyrrolo[2,3-c]pyridyl, 2,3-dihydro-furo[2,3-c]pyridyl, pyrrolo[1,2-a]pyrazinyl, thieno[3,2-c]pyridyl, furo[2,3-c]pyridyl, thieno[2,3-c]pyridyl, furo[3,2-c]pyridyl, 1,1-dioxo-1,3-dihydro-1λ⁶-benzo[d]isothiazol-2-yl, or triazinyl, wherein Het^{(I)} is optionally and independently substituted with from one to three substituents selected from the group consisting of halo, hydroxy, oxo, C₁₋₆alkyl, C₁₋₆alkenyl, C₁₋₆alkynyl, C₁₋₆alkoxy, phenylC₀₋₆alkylenyl-, benzyloxy-carbonyl-, and C₁₋₆alkoxycarbonyl-;
R⁵ is C₁₋₆alkyl or phenylC₀₋₆alkylenyl-;
R⁶ is H, C₁₋₆alkylenyl, or C₃₋₈cycloalkyl;
Het^{(II)} is furanyl, dihydrofuranyl, tetrahydrofuranyl, pyranyl, dihydropyranyl, tetrahydropyranyl, thienyl, dihydrothienyl, tetrahydrothienyl, pyridyl, pyrimidyl, pyrazinyl, pyrrolidinyl, piperidinyl, imidazolyl, pyrazolyl, pyrrolyl, oxazolyl, isoxazolyl, thiazolyl, thiazolidinyl, thiadiazolyl, triazolyl, azetidinyl, dioxanyl, morpholinyl, thiomorpholinyl, imidazolidinyl, thiazolidinyl or a benzo-fused analogue of said Het, wherein Het^{(II)} is substituted with one to three substitutents independently selected from the group consisting of hydroxy, aminocarbonyl-, C₁₋₆alkylaminocarbonyl-, phenyl-C₁₋₆alkylamino carbonyl-, cyano, phenyl-C₁₋₆alkylenylamino-, benzylidene, benzyloxy-C₁₋₆alkylenyl-, benzyloxycarbonyl-, C₁₋₆alkoxycarbonyl-, nitro, and —NR⁷R⁸, and wherein Het^{(II)} is optionally substituted with one to three substituents independently selected from the group consisting of halo, trifluoromethyl, oxo, C₁₋₆alkyl, C₁₋₆alkoxy, C₁₋₆alkylphenyl-, or C₁₋₆alkylcarbonyl; and
R⁷ and R⁸ are each independently selected from H or C₁₋₆alkyl, or R and R⁸ may be taken together with the N atom to which they are attached to form a three to seven membered saturated, partially unsaturated, or unsaturated heterocyclic ring, wherein said heterocyclic ring optionally comprises an additional one to three heteroatoms selected from O, S, and N.

2. The compound of claim 1 further comprising a cyclohexane ring having said ring's 1,4-substituents in the trans stereoconfiguration.

3. The compound of claim 2, wherein:
X is H or —CN;
A is CH₂, CHF, CF₂ or S;
R¹ is —NR²R³, Het^{(I)}, or Het^{(II)};
R² is —C(O)R⁴;
R³ is H;
R⁴ is selected from the group consisting of
(a) Het^{(I)}-C₀₋₆alkylenyl-,
(b) Het^{(II)}-C₀₋₆alkylenyl-, and
(c) R⁵OC(O)N(R⁶)-C₁₋₆alkylenyl-; OK
Het^{(I)} is oxazolidinyl, 6,7-dihydro-5H-pyrrolo[3,4-b]pyrazinyl, 6,7-dihydro-5H-pyrrolo[3,4-b]pyridyl, 2,3-dihydro-1H-pyrrolo[3,4-c]pyridyl, 5,6-dihydro-4H-thieno[2,3-c]pyrrolyl, pyrrolo[1,2-c]pyrimidyl, 1H-pyrrolo[2,3-c]pyridyl, 2,3-dihydro-furo[2,3-c]pyridyl, pyrrolo[1,2-a]pyrazinyl, thieno[3,2-c]pyridyl, furo[2,3-c]pyridyl, thieno[2,3-c]pyridyl, furo[3,2-c]pyridyl, or 1,1-dioxo-1,3-dihydro-1λ⁶-benzo[d]isothiazol-2-yl, wherein Het^{(I)} is optionally and independently substituted with from one to three substituents selected from the group consisting of halo, hydroxy, oxo, C₁₋₆alkyl, C₁₋₆alkenyl, C₁₋₆alkynyl, C₁₋₆alkoxy, phenylC₀₋₆alkylenyl-, benzyloxy-carbonyl-, and C₁₋₆alkoxycarbonyl-;
R⁵ is phenylC₀₋₆alkylenyl-;
R⁶ is H or C₁₋₆alkylenyl;
Het^{(II)} is pyridyl, pyrazinyl, pyrrolidinyl, pyrazolyl, imidazolidinyl or isoindole, wherein Het^{(II)} is substituted with one to three substitutents independently selected from the group consisting of hydroxy, aminocarbonyl-, C₁₋₆alkylaminocarbonyl-, phenyl-C₁₋₆alkylaminocarbonyl-, cyano, phenyl-C₁₋₆alkylenylamino-, benzylidene, benzyloxy-C₁₋₆alkylenyl-, benzyloxycarbonyl-, C₁₋₆alkoxycarbonyl-, nitro, and —NR⁷R⁸, and wherein Het^{(II)} is optionally substituted with one to three substituents independently selected from the group consisting of halo, trifluoromethyl, oxo, C₁₋₆alkyl, C₁₋₆alkoxy, C₁₋₆alkylphenyl-, or C₁₋₆alkylcarbonyl-; and
R⁷ and R⁸ are each independently selected from H or C₁₋₆alkyl.

4. The compound of claim 3 wherein R¹ is —NR²R³.

5. The compound of claim 4 wherein R⁴ is Het^{(II)}-C₀₋₆alkylenyl-.

6. The compound of claim 5 wherein Het^{(II)} is selected from pyrazinyl and pyridyl.

7. The compound of claim 6 wherein Het^{(II)} is substituted with —NR⁷R⁸.

8. The compound of claim 7 wherein R² is

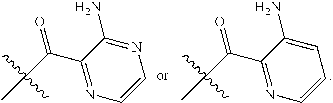

9. The compound of claim 8 wherein A is S.

10. (S)-3-amino-pyrazine-2-carboxylic acid [trans-4-(1-amino-2-oxo-2-thiazolidin-3-yl-ethyl)-cyclohexyl]-amide or a pharmaceutically acceptable salt thereof.

11. A compound of claim 1, 3, 4, 6, 7 or 10, or a prodrug thereof, or a pharmaceutically acceptable salt of said compound or prodrug, or a solvate of said compound, prodrug or salt, for use in therapy.

12. A pharmaceutical composition comprising:
(a) a compound of claim 1, 3, 4, 6, 7 or 10, or a prodrug thereof, or a pharmaceutically acceptable salt of said compound or prodrug, or a solvate of said compound, prodrug or salt; and
(b) a pharmaceutically acceptable carrier, vehicle, diluent or excipient.

13. A method of inhibiting dipeptidyl peptidase-IV in a mammal comprising administering to said mammal in need of such treatment a therapeutically effective amount of a compound of claim 1, 3, 4, 6, 7 or 10, or a prodrug thereof, or a pharmaceutically acceptable salt of said compound or prodrug, or a solvate of said compound, prodrug or salt.

14. A method of treating a condition mediated by dipeptidyl peptidase-IV in a mammal comprising administering to said mammal in need of such treatment a therapeutically effective amount of a compound of claim 1, 3, 4, 6, 7 or 10, or a prodrug thereof, or a pharmaceutically acceptable salt of said compound or prodrug, or a solvate of said compound, prodrug or salt.

15. The method of claim 14 wherein the condition treated is Type 2 diabetes, Type 1 diabetes, impaired glucose tolerance, hyperglycemia, metabolic syndrome (syndrome X and/or insulin resistance syndrome), glucosuria, metabolic acidosis, arthritis, cataracts, diabetic neuropathy, diabetic nephropathy, diabetic retinopathy, diabetic cardiomyopathy, obesity, conditions exacerbated by obesity, hypertension, hyperlipidemia, atherosclerosis, osteoporosis, osteopenia, frailty, bone loss, bone fracture, acute coronary syndrome, short stature due to growth hormone deficiency, infertility due to polycystic ovary syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome; short bowel syndrome; and the prevention of disease progression in Type 2 diabetes.

16. The method of claim 15 wherein the condition treated is Type 2 diabetes.

17. A method of treating diabetes comprising administering to said mammal in need of such treatment a therapeutically effective amount of a compound of claim 1, 3, 4, 6, 7 or 10, or a prodrug thereof, or a pharmaceutically acceptable salt of said compound or prodrug, or a solvate of said compound, prodrug or salt.
